(19) United States
(12) Patent Application Publication
(10) Pub. No.: US 2005/0244364 A1
(43) Pub. Date: Nov. 3, 2005

(54) Novel polymers, compositions comprising them, processes therefor, and use thereof

(76) Inventors: Timo Luukas, Massy (FR); Celine Farcet, Paris (FR)

Correspondence Address: Thomas L. Irving;FINNEGAN, HENDERSON, FARABOW,, GARRETT & DUNNER, L.L.P., 901 New York Avenue, N.W., Washington, DC, 20001-4413, US

(21) Appl. No.: 11/088,908

(22) Filed: Mar. 25, 2005

Related U.S. Application Data
(60) Provisional application No. 60/560,272, filed on Apr. 8, 2004.

(30) Foreign Application Priority Data
Mar. 26, 2004 (FR) 0403184

(51) Int. Cl. A61K 31/785

(52) U.S. Cl. 424/78.3; 526/259

(57) ABSTRACT

The present disclosure relates to novel block polymers comprising at least one monomer with an optical effect, and to cosmetic compositions comprising them, for example nail varnish compositions, foundation compositions, and anti-ageing compositions. The present disclosure also relates to cosmetic treatment processes using the compositions disclosed herein, and to the use of these polymers as tensioning agents.

This application claims benefit of U.S. Provisional Application No. 60/560,272, filed Apr. 8, 2004, and French Application 0403184, filed Mar. 26, 2004, the contents of both of which are incorporated herein by reference.

The present disclosure relates to novel polymers of specific structure and to cosmetic compositions comprising such polymers. The present disclosure also relates to cosmetic treatment processes using the said polymers.

Various types of polymers are conventionally used in cosmetic compositions on account of the various properties that they can give to these compositions. They are used, for example, in makeup and care compositions for the skin, the lips and the integuments, such as nail varnishes and hair care compositions. However, in using two polymers that are incompatible, i.e. immiscible in the same solvent, within the same composition, the formulator is confronted, as a result of the incompatibility of the polymers, with problems of phase separation or even of decantation, and in general with the production of a non-uniform composition. These problems were usually solved until now by the presence in the composition of a compound for rendering the polymers mutually compatible.

Thus, one aim of the present disclosure is to propose polymers which, when included in compositions, for example cosmetic compositions, can enable these compositions to avoid the drawbacks, limitations, defects, and disadvantages of the compositions of the prior art.

In one embodiment, this aim can be achieved, in accordance with the present disclosure, by means of a polymer, referred to as a block polymer, comprising at least a first block and at least a second block that are incompatible with each other, for example having different glass transition temperatures (Tg), wherein the at least first and second blocks are linked together via an intermediate segment comprising at least one constituent monomer of the first block and at least one constituent monomer of the second block.

One aspect of the present disclosure is thus a polymer as defined below.

Another aspect of the present disclosure is a composition, for example a cosmetic composition, comprising at least one polymer as described herein.

It has surprisingly been found that the polymers according to the present disclosure may have good optical properties that make it possible to use them in cosmetics, to obtain adequate optical effects for the compositions comprising them and/or for the makeup comprising these compositions.

Depending on the nature of the substituents, they may show large variability in color, which may range from red to red/violet. This makes it possible to have a range of compounds, belonging to the same chemical family and thus being formulated in a similar manner, that have a diversity of optical properties. This can facilitate the work of formulators by allowing them to keep a common architecture for all of their compositions, irrespective of the polymers with an optical property that are used.

Moreover, the polymers according to the present disclosure can have good fluorescence properties. It is known that fluorescent compounds absorb in the ultraviolet and visible range, and re-emit energy by fluorescence with a wavelength ranging from 380 nm to 830 nm.

The polymers according to the present disclosure may be in solid or liquid form, and give noteworthy optical effects to the compositions comprising them and also to the makeup applied. For example, they can bring lightening effects, illuminating effects and/or color effects.

In one embodiment, these polymers show good temperature, pH, and light stability.

It has also surprisingly been found that the polymers according to the present disclosure can show good solubility in fatty substances, and that this solubility can vary and be adjusted according to the nature of the monomers. This good liposolubility may also facilitate their subsequent use, for example in cosmetic compositions, which generally comprise a fatty phase.

The term “at least one block,” as used herein, is understood to mean one or more blocks. As used herein, the term “monomer” also includes monomeric units when, for example, referring to the units of a polymer obtained following polymerization.

The term “mutually incompatible-blocks,” as used herein, is understood to mean that the mixture formed from the polymer corresponding to the at least one first block and from the polymer corresponding to the at least one second block is immiscible in the polymerization solvent that is in weight majority for the block polymer, at room temperature (25° C.) and atmospheric pressure (10⁵ Pa), for a polymer mixture content of greater than or equal to 5% by weight, relative to the total weight of the mixture (polymers and solvent), it being understood that:
i) the polymers are present in the mixture in a content such that the respective weight ratio ranges from 10/90 to 90/10, and that
ii) each of the polymers corresponding to the at least one first and second blocks has an average (weight-average or number-average) molecular mass equal to that of the block polymer ±15%.

In the case where two or more polymerization solvents are present, the polymer mixture is immiscible in at least one of them.

Needless to say, in the case of a polymerization performed in a single solvent, this solvent is the solvent that is in majority.

The intermediate segment is a block comprising at least one constituent monomer ml of the first block and at least one constituent monomer m2 of the second block of the polymer. For example, m2 may be different from m1. The intermediate segment or block may allow these first and second blocks to be “compatibilized”.

By incorporating these novel polymers into cosmetic compositions, the Applicants have discovered that some of these polymers described in greater detail hereinbelow can have very beneficial cosmetic properties.

In general, these polymers may increase the impact strength of nail varnishes and may improve the staying power of a wide variety of makeup compositions, for example foundations or lipsticks, without causing the user any sensation of discomfort. They may moreover have tensioning properties.

The block polymers of the compositions according to the present disclosure can be, for example, linear block ethylenic polymers, such as those that form a deposit, for instance film-forming polymers.

The term “ethylenic polymer,” as used herein, is understood to mean a polymer obtained by polymerization of monomers comprising an ethylenic unsaturation.

The term “block polymer,” as used herein, is understood to mean a polymer comprising at least two different blocks, for example at least 3 different blocks.

The polymer is a polymer of linear structure. In contrast, a polymer of non-linear structure is, for example, a polymer of branched, starburst or grafted structure, or the like.

The term “polymer forming a deposit,” as used herein, is understood to mean a polymer capable, by itself or in the presence of an auxiliary agent, of forming a deposit that adheres to a support, for example to keratin materials.

The term “film-forming polymer,” as used herein, is understood to mean a polymer that is capable, by itself or in the presence of an auxiliary film-forming agent, of forming a continuous film that adheres to a support, for example to keratin materials.

The polymers according to the present disclosure comprise at least one first block and at least one second block that are incompatible with each other, for example having different glass transition temperatures (Tg), the at least one first and second blocks being linked together via an intermediate segment comprising at least one constituent. monomer of the at least one first block and at least one constituent monomer of the at least one second block.

It is pointed out that, in the text hereinabove and hereinbelow, the terms “first” and “second” blocks do not in any way condition the order of the blocks in the structure of the polymers.

Each block of the polymers according to the present disclosure is derived from one type of monomer or from several different types of monomer. This means that each block may comprise a homopolymer or a copolymer. The copolymer comprised in the block may in turn be random or alternating.

For example, the intermediate segment comprising at least one constituent monomer of the at least one first block and at least one constituent monomer of the at least one second block of the polymers can be a random polymer. For instance, the intermediate block can essentially be derived from constituent monomers of the at least one first block and of the at least one second block. The term “essentially,” as used herein, is understood to mean at least 85%, for example at least 90%, for instance 95%, such as 100%.

For example, the intermediate block can have a glass transition temperature Tg that is between the glass transition temperatures of the at least one first and second blocks.

According to the present disclosure, the at least one first and second blocks may have different glass transition temperatures, with a difference between the glass transition temperatures of the at least one first and second blocks greater than 5° C., for example greater than 10° C., such as greater than 20° C.

The glass transition temperatures indicated for the at least one first and second blocks may be theoretical Tg values determined from the theoretical Tg values of the constituent monomers of each of the blocks, which may be found in a reference manual such as the Polymer Handbook, 3rd Edition, 1989, John Wiley, according to the following relationship, known as Fox's law:

$$\frac{1}{Tg} = {\sum }_{i} ( \frac{\overline{ω} i}{Tgi} )$$

wherein \overline{ω}i is the mass fraction of the monomer i in the block under consideration and Tgi is the glass transition temperature of the homopolymer of the monomer i.

Unless otherwise indicated, the Tg values indicated for the at least one first and second blocks in the present disclosure are theoretical Tg values.

In one embodiment, at least one of the blocks comprises at least one monomer, hereinbelow referred to as a monomer “with an optical effect,” chosen from those of formula (I): 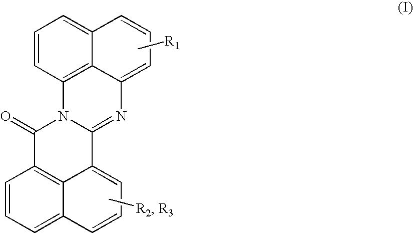
wherein:
R₁ is chosen from hydrogen; halogen atoms; linear, branched and/or cyclic, saturated and/or unsaturated carbon-based radicals comprising from 1 to 12 carbon atoms optionally substituted with at least one substituent chosen from ═O, OH, and NH₂, and/or optionally interrupted with at least one heteroatom chosen from O, N, P, Si, and S; and groups of formula NRR′, wherein R and R′, which may be identical or different, are chosen from hydrogen and linear, cyclic and branched, saturated hydrocarbon-based radicals comprising from 1 to 6 carbon atoms, for example methyl, ethyl, propyl, isopropyl, n-butyl, isobutyl, tert-butyl, pentyl and hexyl; and
R₂ and R₃, which may be present on the same ring or on a different ring, and which may be identical or different, are chosen from hydrogen, halogen atoms, and groups of formula (II), with the proviso that at least one of the radicals R₂ and R₃ is chosen from groups of formula (II):
   —X-G-P (II)
   wherein:
X is chosen from the entities —O—, —S—, —SO—, —SO₂—, —NH—, and —NR″—, wherein
R″ is chosen from linear, branched and/or cyclic, saturated and/or unsaturated carbon-based radicals comprising from 1 to 30 carbon atoms, optionally substituted with at least one substituent chosen from ═O, OH, NH₂, and halogen atoms, and/or optionally interrupted with at least one heteroatom chosen from O, N, P, Si, and S;
G is chosen from linear, branched and/or cyclic, saturated and/or unsaturated divalent carbon-based radicals comprising from 1 to 32 carbon atoms, optionally substituted with at least one substituent chosen from ═O, OH, NH₂, and halogen atoms, and/or optionally interrupted with at least one heteroatom chosen from O, N, P, Si, and S;
P is a polymerizable group chosen from those of formulae (IIIa), (IIIb), and (IIIc): 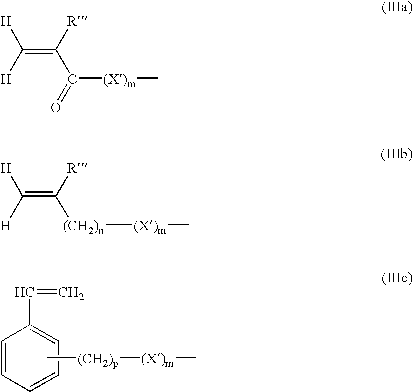
   wherein:
R′″ is chosen from hydrogen and linear and branched, saturated C₁-C₆ hydrocarbon-based radicals;
X′ is chosen from O, NH, and NR″″, wherein R″″ is chosen from C₁-C₆ alkyl, C₆-C₁₀ aryl, (C₆-C₁₀)aryl(C₁-C₆)alkyl, and (C₁-C₆)alkyl(C₆-C₁₀)aryl radicals, wherein the alkyl and/or aryl groups may be substituted with at least one substituent chosen from OH, halogen, and C₁-C₆ alkoxy, and C₆-C₁₀ aryloxy radicals; for example, X′ may be O;
m is equal to 0 or 1;
n is equal to 0 or 1; and
p is equal to 0, 1 or 2.

In the present disclosure, the term “cyclic radical” is understood to mean a monocyclic or polycyclic radical, which itself is in the form of at least one saturated and/or unsaturated, optionally substituted ring (for example cyclohexyl, cyclodecyl, benzyl or fluorenyl), but also a radical comprising at least one such ring (for example 4-hydroxybenzyl).

In the present disclosure, the term “saturated and/or unsaturated radical” is understood to mean totally saturated radicals, totally unsaturated radicals, including aromatic radicals, and also radicals comprising at least one double and/or triple bond, the rest of the bonds being single bonds.

R₁ may be, for example, hydrogen.

R₂ may be, for example, hydrogen, in which case R₃ is a group of formula (II).

In formula (II), X may be chosen from, for example, —O— and —NR″—, wherein R″ is chosen from, for example, linear, branched and/or cyclic, saturated and unsaturated hydrocarbon-based radicals, optionally comprising a hydrocarbon-based ring that is itself saturated or unsaturated, said hydrocarbon-based radicals comprising from 2 to 18, such as from 3 to 12, carbon atoms, and said hydrocarbon-based radicals being optionally substituted with at least one substituent chosen from ═O, OH, NH₂, and halogen atoms, and/or being optionally interrupted with at least one heteroatom chosen from O, N, P, Si, and S.

R″ may be chosen from, for example, ethyl, n-propyl, isopropyl, n-butyl, isobutyl, tert-butyl, pentyl, hexyl, cyclohexyl, octyl, cyclooctyl, decyl, cyclodecyl, dodecyl, cyclodecyl, phenyl, and benzyl radicals.

The divalent radical G may be chosen from, for example, linear, branched and/or cyclic, saturated and unsaturated divalent hydrocarbon-based radicals, optionally comprising a hydrocarbon-based ring that is itself saturated or unsaturated, said hydrocarbon-based radicals comprising in total from 2 to 18, such as from 3 to 10 carbon atoms, and said hydrocarbon-based radicals being optionally substituted with at least one substituent chosen from ═O, OH, NH₂, and halogen atoms, and/or being optionally interrupted with at least one heteroatom chosen from O, N, P, and Si.

For instance, G may be chosen from linear and branched, saturated divalent hydrocarbon-based radicals optionally comprising a saturated hydrocarbon-based ring, said hydrocarbon-based radicals comprising in total from 2 to 18, such as from 3 to 10 carbon atoms.

Thus, G may be chosen from ethylene, n-propylene, isopropylene (or 1-methylethylene and 2-methylethylene), n-butylene, isobutylene, pentylene, for example n-pentylene, hexylene, for example n-hexylene, cyclohexylene, heptylene, octylene, cyclooctylene, decylene, cyclodecylene, cyclohexyldimethylene, for example of formula —CH₂—C₆H₁₀—CH₂—, dodecylene, and cyclododecylene radicals.

In the formula (IIIb), if n equals 0, then m may, for example, equal 0.

The polymerizable group P may be chosen, for example, from one of the following formulae: 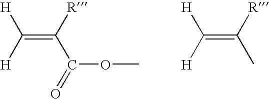
wherein R′″ is chosen from H and methyl.

Among the monomer compounds that may be used according to the present disclosure, non-limiting mention may be made of the compounds corresponding to one of the following formulae, wherein R is hydrogen or methyl:

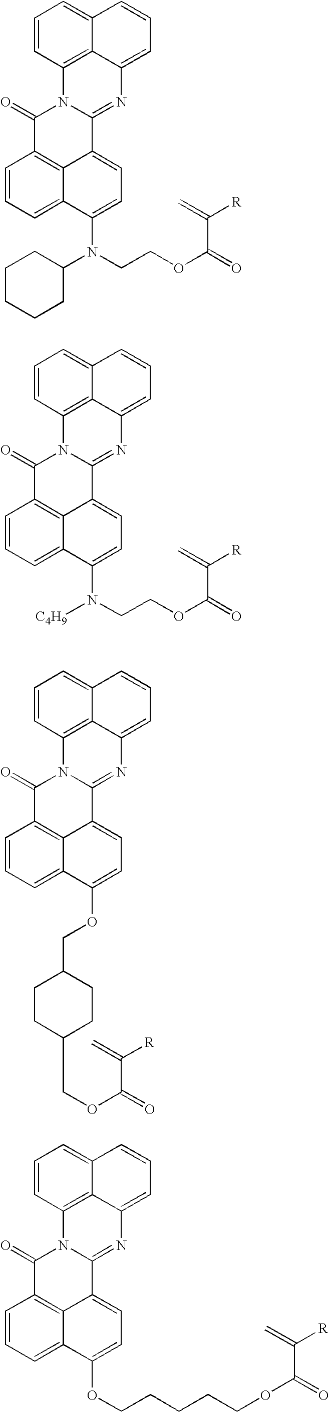

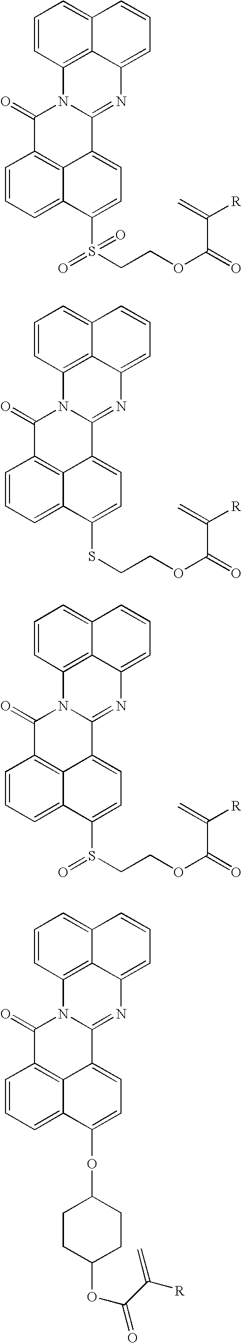

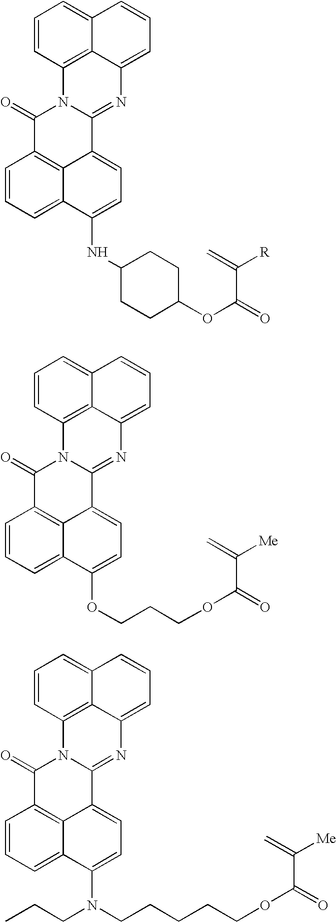

The at least one monomer may be present, alone or as a mixture, in the first and/or the second block.

The block comprising the at least one monomer with an optical effect of formula (I) may thus be chosen from:
(i) a homopolymer comprising only one monomer with an optical effect of formula (I),
(ii) a copolymer comprising several monomers with an optical effect of formula (I),
(iii) a copolymer comprising at least one monomer with an optical effect of formula (I), and at least one additional monomer, which may be chosen, for example, from the monomers with an optical effect of formula (A), (B) and/or (C) below and the “usual” additional monomers.

Among the additional monomers that may be present in the block comprising the at least one monomer with an optical effect of formula (I), and/or which may be present in the other block(s) not comprising a monomer with an optical effect of formula (I), non-limiting mention may be made, alone or as a mixture, of the following monomers:
(i) ethylenic hydrocarbons comprising from 2 to 10 carbons, such as ethylene, isoprene, and butadiene;
(ii) the (meth)acrylates of formulae: 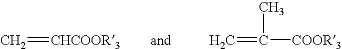
   wherein R′₃ may be chosen from:
linear and branched alkyl groups comprising from 1 to 18 carbon atoms, optionally intercalated with at least one heteroatom chosen from O, N, S, and P, and optionally substituted with at least one substituent chosen from hydroxyl groups, halogen atoms (Cl, Br, I, and F), and groups of formula Si(R₄R₅), wherein R₄ and R₅, which may be identical or different, are chosen from C₁ to C₆ alkyl groups and phenyl groups;
for example, R′₃ may be chosen from methyl, ethyl, propyl, n-butyl, isobutyl, tert-butyl, hexyl, ethylhexyl, octyl, lauryl, isooctyl, isodecyl, dodecyl, cyclohexyl, t-butylcyclohexyl, and stearyl groups; 2-ethylperfluorohexyl; C₁-C₄ hydroxyalkyl groups such as 2-hydroxyethyl, 2-hydroxybutyl, and 2-hydroxypropyl; and (C₁-C₄)alkoxy(C₁-C₄)alkyl groups such as methoxyethyl, ethoxyethyl, and methoxypropyl;
C₃ to C₁₂ cycloalkyl groups such as isobornyl groups;
C₃ to C₂₀ aryl groups such as phenyl groups;
C₄ to C₃₀ aralkyl groups (C₁ to C₈ alkyl groups) such as 2-phenylethyl, t-butylbenzyl, and benzyl groups;
4- to 12-membered heterocyclic groups comprising at least one heteroatom chosen from O, N, and S, the ring being aromatic or non-aromatic;
—heterocycloalkyl groups (C₁ to C₄ alkyl groups), such as furfurylmethyl and tetrahydrofurfurylmethyl groups;
   wherein the cycloalkyl, aryl, aralkyl, heterocyclic, and heterocycloalkyl groups may optionally be substituted with at least one substituent chosen from hydroxyl groups, halogen atoms, and linear and branched C₁-C₄ alkyl groups optionally intercalated with at least one heteroatom chosen from O, N, S, and P, the alkyl groups also possibly being optionally substituted with at least one substituent chosen from hydroxyl groups, halogen atoms (Cl, Br, I and F), and groups of formula Si(R₄R₅), wherein R₄ and R₅, which may be identical or different, are chosen from C₁ to C₆ alkyl groups and phenyl groups; and
groups of formula —(C₂H₄O)_{q}—R₁₃, wherein q ranges from 5 to 150 and R₁₃ is chosen from hydrogen and C₁ to C₃₀ alkyl groups, for example —POE-methyl and —POE-behenyl groups;
(iii) the (meth)acrylamides of formula: 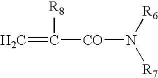
   wherein:
R₈ is chosen from hydrogen and methyl; and
R₆ and R₇, which may be identical or different, are chosen from:
   hydrogen;
   linear and branched alkyl groups comprising from 1 to 18 carbon atoms, optionally intercalated with at least one heteroatom chosen from O, N, S, and P, and optionally substituted with at least one substituent chosen from hydroxyl groups, halogen atoms (Cl, Br, I and F), and groups of formula Si(R₄R₅), wherein R₄ and R₅, which may be identical or different, are chosen from C₁ to C₆ alkyl groups and phenyl groups;
   R₆ and/or R₇ may, for example, be chosen from methyl, ethyl, propyl, n-butyl, isobutyl, tert-butyl, hexyl, ethylhexyl, octyl, lauryl, isooctyl, isodecyl, dodecyl, cyclohexyl, t-butylcyclohexyl, and stearyl groups; 2-ethylperfluorohexyl; C₁-C₄ hydroxyalkyl groups such as 2-hydroxyethyl, 2-hydroxybutyl, and 2-hydroxypropyl; and (C₁-C₄)alkoxy(C₁-C₄)alkyl groups such as methoxyethyl, ethoxyethyl, and methoxypropyl;
   C₃ to C₁₂ cycloalkyl groups, such as isobornyl groups;
   C₃ to C₂₀ aryl groups, such as phenyl groups;
   C₄ to C₃₀ aralkyl groups (C₁ to C₈ alkyl groups), such as 2-phenylethyl, t-butylbenzyl, and benzyl groups;
   4- to 1 2-membered heterocyclic groups comprising at least one heteroatom chosen from O, N, and S, the ring being aromatic or non-aromatic; and
   heterocycloalkyl groups (C₁ to C₄ alkyl groups), such as furfurylmethyl and tetrahydrofurfurylmethyl groups;
      wherein the cycloalkyl, aryl, aralkyl, heterocyclic, and heterocycloalkyl groups may optionally be substituted with at least one substituent chosen from hydroxyl groups, halogen atoms, and linear and branched C₁-C₄ alkyl groups optionally intercalated with at least one heteroatom chosen from O, N, S, and P, the alkyl groups also possibly being optionally substituted with at least one substituent chosen from hydroxyl groups, halogen atoms (Cl, Br, I and F), and groups of formula Si(R₄R₅), wherein R₄ and R₅, which may be identical or different, are chosen from C₁ to C₆ alkyl groups and phenyl groups.

Non-limiting examples of (meth)acrylamide monomers include (meth)acrylamide, N-ethyl(meth)acrylamide, N-butylacrylamide, N-t-butylacrylamide, N-isopropylacrylamide, N,N-dimethyl(meth)acrylamide, N,N-dibutylacrylamide, N-octylacrylamide, N-dodecylacrylamide, undecylacrylamide, and N(2-hydroxypropylmethacrylamide).
(iv) the vinyl compounds chosen from those of formulae:
   CH₂═CH—R₉, CH₂═CH—CH₂—R₉, and CH₂═C(CH₃)—CH₂—R₉
   wherein R₉ is chosen from hydroxyl groups; halogen atoms (Cl or F); NH₂; groups of formula OR₁₀, wherein R₁₀ is chosen from phenyl groups and C₁ to C₁₂ alkyl groups (the monomer is a vinyl or allylic ether); acetamide (NHCOCH₃); groups of formula OCOR₁₁, wherein R₁₁ is chosen from linear and branched alkyl groups comprising from 2 to 12 carbon atoms (the monomer is a vinyl or allylic ester); and groups chosen from:
linear and branched alkyl groups comprising from 1 to 18 carbon atoms, optionally intercalated with at least one heteroatom chosen from O, N, S, and P, and optionally substituted with at least one substituent chosen from hydroxyl groups, halogen atoms (Cl, Br, I and F), and groups of formula Si(R₄R₅), wherein R₄ and R₅, which may be identical or different, are chosen from C₁ to C₆ alkyl groups and phenyl groups;
C₃ to C₁₂ cycloalkyl groups, such as isobornyl and cyclohexane groups;
C₃ to C₂₀ aryl groups, such as phenyl groups;
C₄ to C₃₀ aralkyl groups (C₁ to C₈ alkyl groups), such as 2-phenylethyl, t-butylbenzyl, and benzyl groups;
4- to 12-membered heterocyclic groups comprising at least one heteroatom chosen from O, N, and S, the ring being aromatic or non-aromatic; and
heterocycloalkyl groups (C₁ to C₄ alkyl groups), such as furfurylmethyl and tetrahydrofurfurylmethyl groups;
   wherein the cycloalkyl, aryl, aralkyl, heterocyclic, and heterocycloalkyl groups may optionally be substituted with at least one substituent chosen from hydroxyl groups, halogen atoms, and linear and branched C₁ to C₄ alkyl groups optionally intercalated with at least one heteroatom chosen from O, N, S, and P, and optionally substituted with at least one substituent chosen from hydroxyl groups, halogen atoms (Cl, Br, I, and F), and groups of formula Si(R₄R₅), wherein R₄ and R₅, which may be identical or different, are chosen from C₁ to C₆ alkyl groups and phenyl groups.

Non-limiting examples of vinyl monomers include vinyl cyclohexane and styrene. Non-limiting examples of vinyl esters include vinyl acetate, vinyl propionate, vinyl butyrate, vinyl ethylhexanoate, vinyl neononanoate, and vinyl neododecanoate.

Among the vinyl ethers, non-limiting mention may be made of methyl vinyl ether, ethyl vinyl ether, and isobutyl vinyl ether.
(v) (meth)acrylic, (meth)acrylamide, and vinyl monomers comprising a fluoro or perfluoro group, such as ethylperfluorooctyl and 2-ethylperfluorohexyl (meth)acrylate;
(vi) silicone-based (meth)acrylic, (meth)acrylamide, and vinyl monomers, such as methacryloxypropyltris(trimethylsiloxy)silane and acryloxypropylpolydimethylsiloxane;
(vii) ethylenically unsaturated monomers comprising at least one functional group chosen from carboxylic, phosphoric, and sulfonic acid, and anhydride functional groups, for instance acrylic acid, methacrylic acid, crotonic acid, maleic anhydride, itaconic acid, fumaric acid, maleic acid, acrylamidopropanesulfonic acid, vinylbenzoic acid, vinylphosphoric acid, and the salts thereof; and
(viii) ethylenically unsaturated monomers comprising at least one tertiary amine functional group, for instance 2-vinylpyridine, 4-vinylpyridine, dimethylaminoethyl methacrylate, diethylaminoethyl methacrylate, dimethylaminopropylmethacrylamide, and the salts thereof.

The salts may be formed by neutralization of anionic groups with a mineral base, such as LiOH, NaOH, KOH, Ca(OH)₂, NH₄OH or Zn(OH)₂; or with an organic base such as a primary, secondary or tertiary alkylamine, for example triethylamine or butylamine. This primary, secondary or tertiary alkylamine may comprise at least one nitrogen and/or oxygen atom and may thus comprise, for example, at least one alcohol functional group. Non-limiting mention may be made of amino-2-methyl-2-propanol, triethanolamine, and dimethylamino-2-propanol. Further non-limiting mention may also be made of lysine and 3-(dimethylamino)propylamine.

Non-limiting mention may also be made of the salts of mineral acids, such as sulfuric acid, hydrochloric acid, hydrobromic acid, hydriodic acid, phosphoric acid or boric acid. Further non-limiting mention may also be made of the salts of organic acids, which may comprise at least one carboxylic, sulfonic or phosphonic acid group. They may be linear, branched, or cyclic aliphatic acids, or alternatively aromatic acids. These acids may also comprise at least one heteroatom chosen from O and N, for example in the form of hydroxyl groups. Non-limiting mention may be made, for example, of propionic acid, acetic acid, terephthalic acid, citric acid, and tartaric acid.

For example, the additional comonomers may be chosen, alone or as a mixture, from C₁-C₁₈ alkyl and C₃-C₁₂ cycloalkyl (meth)acrylates, for instance from methyl acrylate, methyl methacrylate, isobornyl acrylate, isobornyl methacrylate, isobutyl acrylate, isobutyl methacrylate, 2-ethylhexyl acrylate, 2-ethylhexyl methacrylate, dodecyl acrylate, dodecyl methacrylate, stearyl acrylate, stearyl methacrylate, trifluoroethyl acrylate, and trifluoroethyl methacrylate.

Non-limiting mention may also be made of acrylic acid, methacrylic acid, methacryloxypropyltris(trimethylsiloxy)silane, acryloxypropyltris(trimethylsiloxy)silane, acryloxypropylpolydimethylsiloxane, and methacryloxypropylpolydimethylsiloxane.

Among the additional monomers that may be present in the block comprising the at least one monomer with an optical effect of formula (I), and/or that may be present in the other block(s) not comprising a monomer with an optical effect of formula (I), non-limiting mention may be made of the monomers with an optical effect of formulae (A), (B) and/or (C): 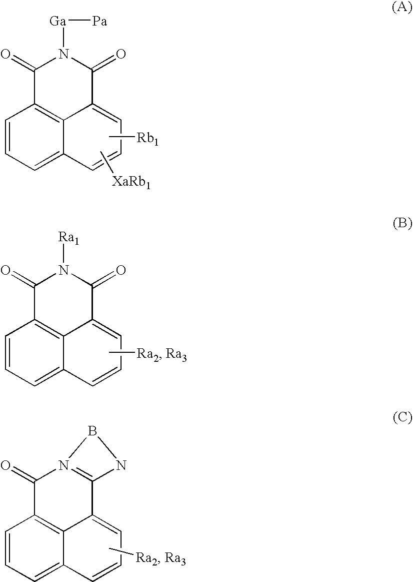
wherein:
Ra₁ is chosen from linear, branched and/or cyclic, saturated and/or unsaturated carbon-based radicals comprising from 1 to 32 carbon atoms, optionally substituted with at least one group chosen from ═O, OH, NH₂, and halogen atoms, and/or optionally interrupted with at least one heteroatom chosen from O, N, P, Si, and S;
Rb₁ is chosen from hydrogen; halogen atoms; linear, branched and/or cyclic, saturated and/or unsaturated carbon-based radicals comprising from 1 to 12 carbon atoms, optionally substituted with at least one group chosen from ═O, OH, and NH₂, and/or optionally interrupted with at least one heteroatom chosen from O, N, P, Si, and S; groups of formula NRR′, wherein R and R′, which may be identical or different, are chosen from hydrogen and linear, cyclic, and branched, saturated C₁ to C₆ hydrocarbon-based radicals, for example methyl, ethyl, propyl, isopropyl, n-butyl, isobutyl, tert-butyl, pentyl, and hexyl;
Ra₂ and Ra₃, which may be present on the same ring or on a different ring, and which may be identical or different, are chosen from hydrogen, halogen atoms, and groups of formula (III), with the proviso that at least one of the radicals Ra₂ and Ra₃ is chosen from groups of formula (III):
   —Xa-Ga-Pa (III)
   wherein:
Xa is chosen from the groups —O—, —S—, —SO—, —SO₂—, —NH—, and —NR″—, wherein R″ is chosen from linear, branched and/or cyclic, saturated and/or unsaturated carbon-based radicals comprising from 1 to 30 carbon atoms, optionally substituted with at least one substituent chosen from ═O, OH, NH₂, and halogen atoms, and/or optionally interrupted with at least one heteroatom chosen from O, N, P, Si, and S;
Ga is chosen from linear, branched and/or cyclic, saturated and/or unsaturated divalent carbon-based radicals comprising from 1 to 32 carbon atoms, optionally substituted with at least one substituent chosen from ═O, OH, NH₂, and halogen atoms, and/or optionally interrupted with at least one heteroatom chosen from O, N, P, Si, and S;
Pa is a polymerizable group chosen from those of formulae (IIIa), (IIIb), and (IIIc): 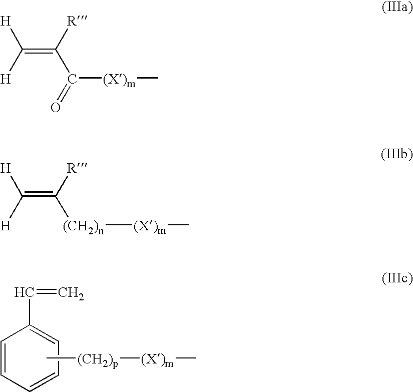
   wherein:
R′″ is chosen from hydrogen and linear and branched, saturated C₁-C₆ hydrocarbon-based radicals,
X′ is chosen from O, NH, and NR″″, wherein R″″ is chosen from C₁-C₆ alkyl, C₆-C₁₀ aryl, (C₆-C₁₀)aryl (C₁-C₆)alkyl, and (C, -C₆)alkyl(C₆-C₁₀)aryl radicals, wherein the alkyl and/or aryl groups may be substituted with at least one group chosen from OH, halogen, and C₁-C₆ alkoxy and C₆-C₁₀ aryloxy radicals;
m is equal to 0 or 1;
n is equal to 0 or 1; and
p is equal to 0, 1 or 2; and
B is chosen from divalent aromatic groups of formulae (IVa) to (IVd): 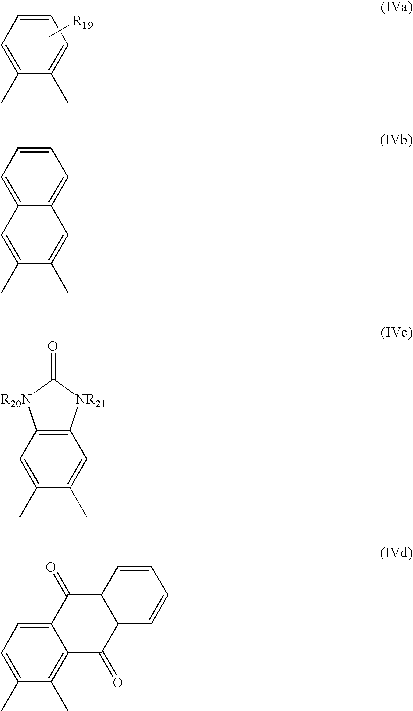
   wherein:
R₁₉ is chosen from linear, branched and/or cyclic, saturated and/or unsaturated carbon-based radicals comprising 1 to 32 carbon atoms, optionally substituted with at least one group chosen from ═O, OH, NH₂, and halogen atoms; and
R₂₀ and R₂₁, which may be identical or different, are chosen from hydrogen, linear and branched C₁₋₈ alkyl radicals, and cyclopentyl, cyclohexyl, cyclooctyl, cyclodecyl, cyclododecyl, benzyl, naphthyl, and phenyl radicals.

For example, the at least one monomer with an optical effect of formula (I) may be present in an amount ranging from 0.01% to 100% by weight, for example from 0.1% to 99.99% by weight, for instance from 0.5% to 70% by weight, such as from 1% to 40% by weight, and such as from 1.5% to 30% by weight, relative to the weight of the block comprising it.

For example, the at least one monomer with an optical effect of formula (I) may be present in an amount ranging from 0.01% to 70% by weight, for example from 0.1% to 50% by weight, for instance from 0.5% to 30% by weight, such as from 1% to 20% by weight, relative to the total weight of the polymer.

Each of the blocks of the polymers according to the present disclosure may comprise at least one monomer of formula (I), which may be identical or different depending on the block.

The at least one additional monomer may be present in an amount ranging from 0 to 99.99% by weight, for example 0.01% to 99.9% by weight, for instance from 30% to 99.5% by weight, such as from 60% to 99% by weight, and such as from 70% to 98.5% by weight, relative to the weight of the block comprising it and comprising the monomer(s) with an optical effect of formula (I). Of course, it is present in a proportion of 100% by weight in the possible block(s) not comprising any monomer of formula (I).

The at least one additional monomer may be present in an amount ranging from 30% to 99.99% by weight, for example from 50% to 99.9% by weight, for instance from 70% to 99.5% by weight, such as from 80% to 99% by weight, relative to the total weight of the polymer.

For example, the intermediate block (or segment) may comprise at least one constituent monomer m1 of the first block chosen, for instance, from the additional monomers, and at least one constituent monomer m2 of the second block chosen from the additional monomers other than the monomer m1.

In one embodiment, the block polymers may comprise at least one first block that comprises from 0.5% to 15% by weight, for example from 1% to 10% by weight, of monomer(s) of formula (I) and 85% to 99.5% by weight and from 90% to 99% by weight of additional monomers, the percentages being given relative to the total weight of the said block. The at least one second block may comprise 100% by weight of additional monomers; the intermediate block (or segment) may comprise at least one constituent monomer ml of the first block chosen from the additional monomers, and at least one constituent monomer m2 of the second block chosen from the additional monomers other than the monomer m1.

For example, in the block polymers according to the present disclosure, the at least one first block may be chosen from:
a) a block with a Tg of greater than or equal to 40° C.,
b) a block with a Tg of less than or equal to 20° C.,
c) a block with a Tg of between 20 and 40° C.,
   and the second block may be chosen from a category a), b) or c) different from the first block.

The block comprising the at least one monomer with an optical effect of formula (I) may, for example, have a Tg of greater than or equal to 40° C., such as greater than or equal to 60° C. In this case, the other block(s), if they do not comprise any monomers with an optical effect of formula (I), may have a Tg of less than or equal to 40° C., such as less than or equal to 20° C. If a second block comprises at least one monomer with an optical effect of formula (I), it may, for example, have a Tg of less than or equal to 40° C., such as less than or equal to 20° C.

When the block polymers comprise a block with a Tg of greater than or equal to 40° C., this block may have, for example, a Tg ranging from 40° C. to 1 50° C., for instance from 50° C. to 120° C., such as from 60° C. to 120° C. In this case, it may comprise, for example, in total or in part, monomers whose homopolymers have a Tg in the desired range, for instance greater than or equal to 40° C. It may also comprise monomers with a Tg outside this range. These monomers and their concentration may be chosen in an appropriate manner by a person skilled in the art, for example on the basis of Fox's law, to obtain a block of desired Tg.

Among the monomers whose homopolymers have a glass transition temperature Tg of greater than or equal to 40° C., non-limiting mention may be made of: the methacrylates of formula CH₂═C(CH₃)—COOR₂₂, wherein R₂₂ is chosen from linear and branched unsubstituted alkyl groups comprising from 1 to 4 carbon atoms, such as methyl, ethyl, propyl, and isobutyl groups, and C₄ to C₁₂ cycloalkyl groups, such as isobornyl groups;
the acrylates of formula CH₂═CH—COOR₂₃, wherein R₂₃ is chosen from tert-butyl groups and C₄ to C₁₂ cycloalkyl groups such as isobornyl groups;
the (meth)acrylamides of formula CH₂═CR₂₄—CO—NR₂₅R₂₆, wherein R₂₄ is chosen from hydrogen and CH₃, and R₂₅ and R₂₆, which may be identical or different, are chosen from hydrogen and linear and branched C₁ to C₁₂ alkyl groups, such as n-butyl, t-butyl, isopropyl, isohexyl, isooctyl, and isononyl groups; or alternatively R₂₅ is hydrogen and R₂₆ is chosen from 1,1-dimethyl-3-oxobutyl groups; and
mixtures thereof.

Among the monomers whose homopolymers have a glass transition temperature Tg of greater than or equal to 40° C., further non-limiting mention may be made of methyl methacrylate, ethyl methacrylate, isobutyl methacrylate, tert-butyl (meth)acrylate, (meth)acrylic acid, isobornyl (meth)acrylate, N-butylacrylamide, N-t-butylacrylamide, N-isopropylacrylamide, N,N-dimethylacrylamide, N,N-dibutylacrylamide, and mixtures thereof.

When the block polymers comprise a block with a Tg of less than or equal to 20° C., this block may have, for example, a Tg ranging from −100 to 20° C., for instance from −80° C. to 15° C., such as from −50° C. to 0° C. In this case, it may also comprise, for example, in total or in part, monomers whose homopolymers have a Tg in the desired range, for instance less than or equal to 20° C. It may also comprise monomers with a Tg outside this range. These monomers and their concentration are chosen in an appropriate manner by a person skilled in the art, for example on the basis of Fox's law, to obtain a block of desired Tg.

Among the monomers whose homopolymers have a glass transition temperature Tg of less than or equal to 20° C., non-limiting mention may be made of:
the acrylates of formula CH₂═CHCOOR₂₇, wherein R₂₇ is chosen from linear and branched, unsubstituted C₁ to C₁₂ alkyl groups, with the exception of the tert-butyl group, optionally intercalated with at least one heteroatom chosen from O, N, and S;
the methacrylates of formula CH₂═C(CH₃)—COOR₂₈, wherein R₂₈ is chosen from linear and branched, unsubstituted C₆ to C₁₂ alkyl groups, optionally intercalated with at least one heteroatom chosen from O, N, and S;
the vinyl esters of formula R₂₉—CO—O—CH═CH₂, wherein R₂₉ is chosen from linear and branched C₄ to C₁₂ alkyl groups;
C₄-C₁₂ alkyl vinyl ethers, such as methyl vinyl ether and ethyl vinyl ether;
N—(C₄ to C₁₂ alkyl) acrylamides such as N-octylacrylamide; and
mixtures thereof.

Among the monomers whose homopolymers have a glass transition temperature Tg of less than or equal to 20° C., further non-limiting mention may be made for example of methyl acrylate, ethyl acrylate, isobutyl acrylate, 2-ethylhexyl (meth)acrylate, and mixtures thereof.

When the block polymers comprise a block with a Tg of between 20° C. and 40° C., it may comprise, for example, in total or in part, monomers whose homopolymers have a Tg in the desired range, and among which non-limiting mention may be made of n-butyl methacrylate, cyclohexyl acrylate, cyclododecyl acrylate, neopentyl acrylate, isodecylacrylamide, and mixtures thereof.

In one embodiment, the block polymers according to the present disclosure comprise in at least one block, and, for example, in each of the blocks, at least one monomer chosen from (meth)acrylic acid esters. They may optionally also comprise at least one second monomer chosen from acrylic acid, methacrylic acid, and mixtures thereof.

For example, all the monomers other than the at least one monomer with optical effects of formula (I) may be chosen from (meth)acrylic acid esters and (meth)acrylic acid.

According to another embodiment, the block polymers according to the present disclosure comprise at least one first block with a Tg of greater than or equal to 40° C., for example greater than or equal to 50° C., such as greater than or equal to 60° C., and at least one second block with a Tg of less than or equal to 20° C., for example less than or equal to 10° C., such as less than or equal to 0° C.

For example, the proportion, in the final block polymers, of the block with a Tg of greater than or equal to 40° C. may range from 20% to 95% by weight, for instance from 30% to 80%, such as from 50% to 75% by weight, relative to the weight of the final polymer.

For example, the proportion, in the final block polymers, of the block with a Tg of less than or equal to 20° C. may range from 5% to 80% by weight, for instance from 15% to 50%, such as from 25% to 45% by weight, relative to the weight of the final polymer.

The weight-average mass (Mw) of the block polymers according to the present disclosure may be, for example, less than or equal to 300 000; it may range, for instance, from 35 000 to 200 000, such as from 40 000 to 150 000. The number-average mass (Mn) of the block polymers according to the present disclosure may be, for example, less than or equal to 70 000; it may range, for instance, from 5000 to 60 000, such as from 6000 to 50 000. The weight-average (Mw) and number-average (Mn) molar masses may be determined by gel permeation liquid chromatography (THF solvent, calibration curve established with linear polystyrene standards, refractometric and UV detector).

For example, the polydispersity index of the polymers according to the present disclosure may be greater than 2, for instance ranging from 2 to 9, or greater than or equal to 2.5, for instance ranging from 2.5 to 8, or greater than or equal to 2.8, for instance ranging from 2.8 to 7. The polydispersity index Ip of the polymers is equal to the ratio of the weight-average mass Mw to the number-average mass Mn.

The block polymers according to the present disclosure may have, for example, an absorption wavelength ranging from 300 to 700 nm, for instance from 320 to 650 nm, such as from 350 to 600 nm. It may have, for example, an emission wavelength ranging from 380 to 850 nm, for instance from 400 to 750 nm, such as from 450 to 700 nm.

The block polymers according to the present disclosure may be obtained by solution free-radical polymerization according to the following preparation process:
a portion of the polymerization solvent is introduced into a suitable reactor, and the system is heated until the appropriate temperature for the polymerization (typically between 60° C. and 120° C.) is reached,
once this temperature has been reached, the constituent monomers of the first block are added, in the presence of some of the polymerization initiator,
after a time T corresponding to a maximum degree of conversion, for example 90% conversion, the constituent monomers of the second block and the rest of the initiator are introduced, and
the mixture is left to react for a time T′ (ranging, for example, from 3 to 6 hours), at the end of which the mixture is cooled to room temperature (25° C.) so as to obtain the polymer dissolved in the polymerization solvent.

The term “polymerization solvent,” as used herein, is understood to mean a solvent or a solvent mixture chosen, for example, from ethyl acetate, butyl acetate, C₁-C₆ alcohols such as isopropanol and ethanol, aliphatic alkanes such as isododecane, and mixtures thereof. For example, the polymerization solvent may be a mixture of butyl acetate and isopropanol, or may be isododecane.

In one embodiment, the block polymers according to the present disclosure are not water-soluble, i.e., the polymers are not soluble in water or in a mixture of water and of linear or branched monoalcohols comprising from 2 to 5 carbon atoms, such as ethanol, isopropanol or n-propanol, without a pH modification, at an active material content of at least 1% by weight, at room temperature (25° C.).

The present disclosure also relates to compositions, for example cosmetic compositions, comprising at least one block polymer of specific structure, as described above, in a physiologically acceptable medium, such as a cosmetically acceptable medium.

The at least one block polymer according to the present disclosure may be present, alone or as a mixture, in the compositions as disclosed herein in an amount ranging from 0.01% to 75% by weight, for example from 0.1% to 70% by weight, for instance from 1% to 65% by weight, such as from 3% to 60% by weight, such as from 5% to 50% by weight, and such as from 6% to 25% by weight, relative to the total weight of the composition.

The at least one block polymer may be present in the compositions in dissolved form, for example in water, in an oil or in an organic solvent, or alternatively in the form of an aqueous or organic dispersion.

For example, the at least one block polymer according to the present disclosure may be soluble or dispersible in at least one of the phases of the composition comprising them.

The cosmetic or pharmaceutical compositions according to the present disclosure comprise, in addition to the polymers, a physiologically acceptable medium, for example a cosmetically, dermatologically or pharmaceutically acceptable medium, i.e., a medium that is compatible with keratin materials such as facial or bodily skin, hair, eyelashes, eyebrows, and nails.

The compositions may thus comprise a hydrophilic medium comprising water or a mixture of water and at least one hydrophilic organic solvent, chosen for instance from alcohols, for example linear and branched lower monoalcohols comprising from 2 to 5 carbon atoms, such as ethanol, isopropanol or n-propanol, and polyols, for example glycerol, diglycerol, propylene glycol, sorbitol, and pentylene glycol, and polyethylene glycols, or alternatively hydrophilic C₂ ethers and C₂-C₄ aldehydes.

The water or the mixture of water and hydrophilic organic solvents may be present in the compositions according to the present disclosure in an amount ranging from 0.1% to 99% by weight, for example from 10% to 80% by weight, relative to the total weight of the composition.

The compositions may also be anhydrous.

The compositions may also comprise a fatty phase which may comprise fatty substances that are liquid at room temperature (in general 25° C.) and/or of fatty substances that are solid at room temperature, such as waxes, pasty fatty substances and gums, and mixtures thereof. These fatty substances may be of animal, plant, mineral or synthetic origin. This fatty phase may also comprise lipophilic organic solvents.

As fatty substances that are liquid at room temperature, often referred to as oils, which may be used in the compositions according to the present disclosure, non-limiting mention may be made of: hydrocarbon-based oils of animal origin such as perhydrosqualene; hydrocarbon-based plant oils such as liquid triglycerides of fatty acids of 4 to 10 carbon atoms, for instance heptanoic or octanoic acid triglycerides, or alternatively sunflower oil, maize oil, soybean oil, grapeseed oil, sesame seed oil, apricot oil, macadamia oil, castor oil, avocado oil, caprylic/capric acid triglycerides, jojoba oil, shea butter, linear or branched hydrocarbons of mineral or synthetic origin, such as liquid paraffin and derivatives thereof, petroleum jelly, polydecenes, hydrogenated polyisobutene such as parleam; synthetic esters and ethers, for example of fatty acids, for instance purcellin oil, isopropyl myristate, 2-ethylhexyl palmitate, 2-octyldodecyl stearate, 2-octyl-dodecyl erucate, isostearyl isostearate; hydroxylated esters, for instance isostearyl lactate, octyl hydroxystearate, octyldodecyl hydroxystearate, diisostearyl malate, triisocetyl citrate, and fatty alcohol heptanoates, octanoates and decanoates; polyol esters, for instance propylene glycol dioctanoate, neopentyl glycol diheptanoate and diethylene glycol diisononanoate; and pentaerythritol esters; fatty alcohols comprising from 12 to 26 carbon atoms, for instance octyldodecanol, 2-butyloctanol, 2-hexyldecanol, 2-undecylpentadecanol and oleyl alcohol; partially hydrocarbon-based fluoro oils and/or partially silicone-based fluoro oils; silicone oils, for instance volatile or non-volatile, linear or cyclic polymethylsiloxanes (PDMSs), which are liquid or pasty at room temperature, for instance cyclomethicones, dimethicones, optionally comprising a phenyl group, for instance phenyl trimethicones, phenyltrimethylsiloxydiphenylsiloxanes, diphenylmethyidimethyltrisiloxanes, diphenyl dimethicones, phenyl dimethicones and polymethylphenylsiloxanes; and mixtures thereof.

These oils may be present in an amount ranging from 0.01% to 90%, for example from 0.1% to 85% by weight, relative to the total weight of the composition.

The compositions according to the present disclosure may also comprise at least one physiologically acceptable organic solvent. The at least one solvent may be present in an amount ranging from 0.1% to 90%, for example from 0.5% to 85%, such as from 10% to 80% and such as from 30% to 50% by weight, relative to the total weight of the composition.

Non-limiting mention may be made, for example, in addition to the hydrophilic organic solvents mentioned above, of ketones that are liquid at room temperature such as methyl ethyl ketone, methyl isobutyl ketone, diisobutyl ketone, isophorone, cyclohexanone and acetone; propylene glycol ethers that are liquid at room temperature, such as propylene glycol monomethyl ether, propylene glycol monomethyl ether-acetate, and dipropylene glycol mono-n-butyl ether; short-chain esters (comprising from 3 to 8 carbon atoms in total), such as ethyl acetate, methyl acetate, propyl acetate, n-butyl acetate and isopentyl acetate; ethers that are liquid at 25° C., such as diethyl ether, dimethyl ether or dichlorodiethyl ether; alkanes that are liquid at 25° C., such as decane, heptane, dodecane, isododecane and cyclohexane; aromatic cyclic compounds that are liquid at 25° C., such as toluene and xylene; aldehydes that are liquid at 25° C., such as benzaldehyde and acetaldehyde, and mixtures thereof.

For the purposes of the present disclosure, the term “wax” is understood to mean a lipophilic compound that is solid at room temperature (25° C.), which undergoes a reversible solid/liquid change of state, and which has a melting point of greater than or equal to 25° C., which may be up to 120° C. By bringing the wax to the liquid state (melting), it is possible to make it miscible with the oils possibly present and to form a microscopically homogeneous mixture, but, on returning the temperature of the mixture to room temperature, recrystallization of the wax is obtained in the oils of the mixture. The melting point of the wax may be measured using a differential scanning calorimeter (DSC), for example the calorimeter sold under the name DSC 30 by the company Mettler.

The waxes may be hydrocarbon-based waxes, fluorowaxes, and/or silicone waxes and may be of plant, mineral, animal and/or synthetic origin. For example, the waxes may have a melting point of greater than 30° C., for example greater than 45° C. As examples of waxes that may be used in the compositions of the present disclosure, non-limiting mention may be made of beeswax, camauba wax, candellila wax, paraffin, microcrystalline waxes, ceresin, ozokerite, synthetic waxes, for instance polyethylene waxes and Fischer-Tropsch waxes, and silicone waxes, for instance alkyl or alkoxy dimethicones comprising from 16 to 45 carbon atoms.

The gums may be polydimethylsiloxanes (PDMSs) of high molecular weight, cellulose gums, and polysaccharides, and the pasty substances may be hydrocarbon-based compounds, for instance lanolins and derivatives thereof, or PDMSs.

The nature and amount of the solid substances depend on the desired mechanical properties and textures. As a guide, the compositions may comprise waxes in an amount ranging from 0.1% to 50% by weight, for example from 1% to 30% by weight, relative to the total weight of the composition.

The compositions according to the present disclosure may also comprise, in a particulate phase, at least one pigment and/or at least one nacre and/or at least one filler usually used in cosmetic compositions.

The compositions may also comprise at least one other dyestuff chosen from water-soluble dyes and/or liposoluble dyes that are well known to those skilled in the art.

The term “pigments,” as used herein, is understood to mean white or colored, mineral or organic particles of any shape, which are insoluble in the physiological medium and which are intended to color the compositions.

The term “fillers,” as used herein, is understood to mean colorless or white, mineral or synthetic, lamellar or non-lamellar particles intended to give body or rigidity to the compositions, and/or softness, a matt effect and uniformity to the makeup result.

The term “nacres,” as used herein, is understood to mean iridescent particles of any form, produced, for example, by certain molluscs in their shell, or else synthesized.

The at least one pigment may be present in the compositions according to the present disclosure in an amount ranging from 0.01% to 25% by weight, for example from 3% to 10% by weight, relative to the weight of the final composition. It may be white or colored, and mineral or organic. Non-limiting mention may be made of titanium oxide, zirconium oxide or cerium oxide, and also zinc oxide, iron oxide or chromium oxide, ferric blue, chromium hydrate, carbon black, ultramarines (aluminosilicate polysulfides), manganese pyrophosphate, and certain metallic powders such as silver or aluminium powder. Further non-limiting mention may also be made of the D&C pigments and lakes commonly used to give the lips and the skin a makeup effect, which include calcium, barium, aluminium, strontium or zirconium salts.

The at least one nacre may be present in the compositions according to the present disclosure in an amount ranging from 0.01% to 20% by weight, for example from 3% to 10% by weight, relative to the weight of the final composition. Among the nacres that may be used, non-limiting mention may be made of natural mother-of-pearl, mica coated with titanium oxide, with iron oxide, with natural pigment or with bismuth oxychloride, and also colored titanium mica.

Among the liposoluble or water-soluble dyes that may be present in the compositions, alone or as a mixture, in an amount ranging from 0.001% to 15% by weight, for example from 0.01% to 5% by weight, such as from 0.1% to 2% by weight, relative to the total weight of the composition, non-limiting mention may be made of the disodium salt of Ponceau, the disodium salt of alizarin green, quinoline yellow, the trisodium salt of amaranth, the disodium salt of tartrazine, the monosodium salt of rhodamine, the disodium salt of fuchsin, xanthophyll, methylene blue, cochineal carmine, halo-acid dyes, azo dyes, anthraquinone dyes, copper sulfate, iron sulfate, Sudan brown, Sudan red and annatto, and also beetroot juice and carotene.

The compositions according to the present disclosure may also comprise at least one filler, for example in an amount ranging from 0.01% to 50% by weight, such as from 0.02% to 30% by weight, relative to the total weight of the composition. The fillers may be mineral or organic in any form, platelet-shaped, spherical or oblong. Non-limiting mention may be made of talc, mica, silica, kaolin, polyamide (Nylon®) powders, poly-β-alanine powder and polyethylene powder, powders of tetrafluoroethylene polymers (Teflon®), lauroyllysine, starch, boron nitride, hollow polymer microspheres such as those of polyvinylidene chloride/acrylonitrile, for instance Expancel® (Nobel Industrie) or acrylic acid copolymers (Polytrap® from the company Dow Corning) and silicone resin microbeads (for example Tospearls® from Toshiba), elastomeric polyorganosiloxane particles, precipitated calcium carbonate, magnesium carbonate, magnesium hydrocarbonate, hydroxyapatite, hollow silica microspheres (Silica Beads® from Maprecos), glass or ceramic microcapsules, and metal soaps derived from organic carboxylic acids comprising from 8 to 22 carbon atoms, such as from 12 to 18 carbon atoms, for example zinc, magnesium or lithium stearate, zinc laurate or magnesium myristate.

The compositions may also comprise at least one additional polymer such as a film-forming polymer. According to the present disclosure, the term “film-forming polymer” means a polymer capable, by itself or in the presence of an auxiliary film-forming agent, of forming a continuous film that adheres to a support, for example to keratin materials. Among the film-forming polymers that may be used in the compositions of the present disclosure, non-limiting mention may be made of synthetic polymers, of free-radical type or of polycondensate type, polymers of natural origin and mixtures thereof, for example acrylic polymers, polyurethanes, polyesters, polyamides, polyureas and cellulose-based polymers, for instance nitrocellulose.

The compositions according to the present disclosure may also comprise at least one ingredient commonly used in cosmetics, such as vitamins, thickeners, gelling agents trace elements, softeners, sequestering agents, fragrances, acidifying and basifying agents, preserving agents, sunscreens, surfactants, antioxidants, agents for preventing hair loss, antidandruff agents, propellants, ceramides, and mixtures thereof.

Needless to say, a person skilled in the art will take care to select this or these optional additional compound(s), and/or the amount thereof, such that the beneficial properties of the compositions according to the present disclosure are not, or are not substantially, adversely affected by the addition being considered.

The compositions according to the present disclosure may be in the form of a suspension, a dispersion, for example of oil in water by means of vesicles; an optionally thickened or even gelled aqueous or oily solution; an oil-in-water, water-in-oil or multiple emulsion; a gel or a mousse; an oily or emulsified gel; a dispersion of vesicles, for example of lipid vesicles; a two-phase or multiphase lotion; a spray; a free, compact or cast powder; or an anhydrous paste. These compositions may have the appearance of a lotion, a cream, a salve, a soft paste, an ointment, a mousse, a cast or moulded solid, for example in stick or dish form, or a compacted solid.

A person skilled in the art will be able to choose the appropriate galenical form, and also the method for preparing it, on the basis of his general knowledge, taking into account firstly the nature of the constituents used, such as their solubility in the support, and secondly the intended application of the composition.

The cosmetic compositions according to the present disclosure may be in the form of-a care and/or makeup product for bodily or facial skin, the lips, the nails, the eyelashes, the eyebrows and/or the hair, an antisun product, a self-tanning product, or a hair product for caring for, treating, shaping, making up or coloring the hair.

The cosmetic compositions according to the present disclosure may also be in the form of makeup compositions, for example a complexion product such as a foundation, a makeup rouge or an eyeshadow; a lip product such as a lipstick or a lipcare product; a concealer product; a blusher, a mascara or an eyeliner; an eyebrow makeup product, a lip pencil or an eye pencil; a nail product such as a nail varnish or a nailcare product; a body makeup product; a hair makeup product (hair mascara or hair lacquer).

The compositions may also be in the form of protective or care compositions for the skin of the face, the neck, the hands or the body, for example anti-wrinkle compositions, moisturizing or treating compositions; antisun compositions or artificial tanning compositions.

The compositions may also be in the form of hair products, for example for coloring, holding the hairstyle, shaping the hair, caring for, treating or cleansing the hair, such as shampoos, hairsetting gels or lotions, blow-drying lotions, and fixing and styling compositions such as lacquers or sprays.

In one embodiment of the present disclosure, the compositions are in the form of a nail varnish that may comprise, in addition to the at least one block polymer according to the present disclosure, at least one organic solvent, at least one film-forming polymer, and optionally at least one pigment and/or at least one dye.

In another embodiment of the present disclosure, the compositions are in the form of a foundation that may comprise, in addition to the at least one block polymer according to the present disclosure, at least one oil in a fatty phase, at least one pigment, and optionally an aqueous phase.

In yet another embodiment of the present disclosure, the compositions are in the form of anti-ageing or anti-wrinkle compositions, for example intended to be applied to the face and/or the neck, such as to the wrinkled areas of the face, for example around the eyes.

For example, in one embodiment, it has been found, surprisingly, that the use of block polymers according to the present disclosure make it possible to obtain compositions that may be applied to the skin and that may give an immediate tensioning effect on already-formed wrinkles and/or fine lines; the use of these polymers as tensioning agents may be beneficial, because they may make it possible to form an effective tensioning film with effective rigidity, while at the same time being supple so as to avoid an annoying tautness of keratin materials such as the skin, during the application of a composition comprising such agents.

In this case, the block polymers can be, for example, non-elastomeric and water-insoluble. The term “water-insoluble polymer,” as used herein, is understood to mean that the polymer is not soluble in water or in a mixture of water and of linear or branched C₂-C₅ monoalcohols, for instance ethanol, isopropanol or n-propanol, without pH modification, at an active material content of at least 1% by weight, at room temperature (25° C.).

In this embodiment, the at least one first block may have a Tg of greater than or equal to 85° C., for example ranging from 90° C. to 150° C., such as from 100° C. to 120° C. For example, the block with a Tg of greater than or equal to 85° C. is present in an amount ranging from 50% to 90% by weight, relative to the weight of the final polymer, such as from 60% to 80% by weight, relative to the weight of the final polymer. The at least one second block may have a Tg of less than or equal to 20° C., for example ranging from −100° C. to 20° C., such as from −80° C. to 15° C., for instance from −70° C. to 10° C. For example, the block with a Tg of less than or equal to 20° C. may be present in an amount ranging from 5% to 50% by weight, relative to the weight of final polymer, such as from 10% to 40% by weight, relative to the weight of the final polymer.

The compositions according to the present disclosure may also comprise at least one anti-ageing active agent chosen, for example, from desquamating agents, moisturizers, agents for stimulating keratinocyte proliferation and/or differentiation, agents for stimulating collagen and/or elastin synthesis or for preventing their degradation, depigmenting agents, anti-glycation agents, agents for stimulating glycoaminoglycan synthesis, dermo-decontracting agents or muscle relaxants, antioxidants and free-radical scavengers, and mixtures thereof.

Another aspect of the present disclosure is the use of the block polymers as disclosed herein as tensioning agents in cosmetic compositions, for example in an anti-wrinkle compositions.

Another aspect of the present disclosure is a cosmetic process for treating wrinkled skin, such as the contour of the eyes, comprising applying to skin a cosmetic composition comprising, in a cosmetically acceptable medium, at least one block polymer as defined above.

Yet another aspect of the present disclosure is a cosmetic treatment process, for example for making up or caring for keratin materials, such as bodily or facial skin, lips, nails, eyelashes, eyebrows and/or hair, comprising applying to the keratin materials a cosmetic composition as defined above.

The present disclosure may be understood more clearly with the aid of the non-limiting examples that follow, which constitute various embodiments of the compositions according to the disclosure. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements.

Method for Measuring the Wavelength (Emission and Absorption)

Wavelength measurements were performed using a Varian Cary Eclipse fluorimeter. Unless otherwise indicated, these measurements were performed in the following manner:

20 mg of product were placed in a 50 ml cylinder. To dissolve the product, the cylinder was filled to 50 ml with a suitable solvent, for example dichloromethane (DCM), chloroform, isododecane, heptane or dimethyl sulfoxide (DMSO). The resulting solution was mixed and 250 microliters were taken and placed in a 50 ml cylinder, which was then filled to 50 ml again with the solvent.

The whole was mixed and a sample of the solution was taken and placed in a closed quartz cuvette 10 mm thick, which was then placed in the measuring chamber.

### EXAMPLE 1

### Step 1: Preparation of 4-chloronaphthylisoquinolinone

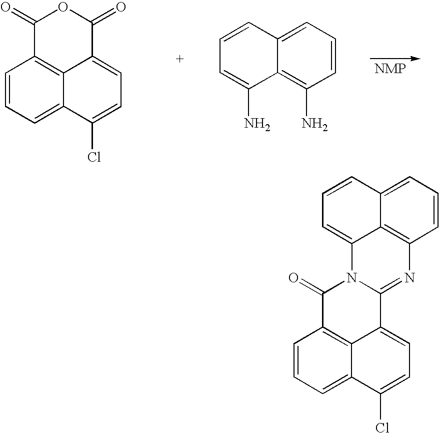

20.0 g (86 mmol) of 4-chloronaphthalene-1,8-anhydride were placed in a 2 liter round-bottomed flask under an inert atmosphere (nitrogen), and 20.4 g (0.13 mol) of 1,8-diaminonaphthalene and 250 ml of NMP were then added. The mixture was stirred at 500 rpm for a few minutes, under argon, and was then heated to 150° C. After reaction for 4 hours, the resulting reaction mixture was allowed to cool to room temperature. The precipitate was filtered off, washed with ethanol and with water, and then oven-dried at 60° C. under reduced pressure.

30.2 g of a brown powder were obtained (98.9% yield).

Characterization

¹H NMR (CDCl₃, 400 MHz) δ:8.84-8.86 (1H), 8.61-8.45 (2H), 8.43-8.37 (1H), 7.79-7.70 (2H), 7.51-7.26 (4H), 7.22-7.18 (1H).

### Step 2: Preparation of Naphthylisoguinolinone-4-aminoethanol

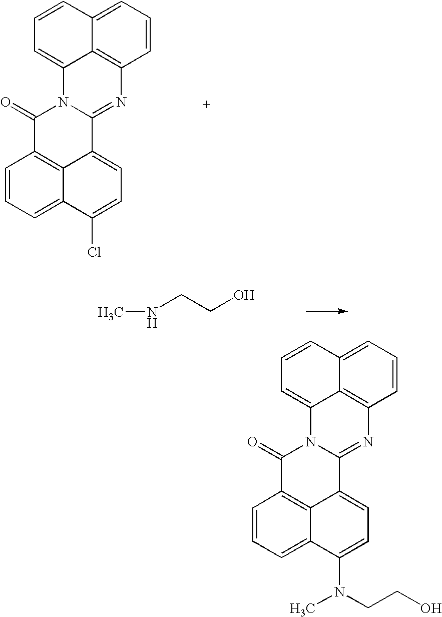

10.0 g (28.8 mmol) of 4-chloronaphthylisoquinolinone were placed in a round-bottomed flask under an inert atmosphere of argon. 120.0 g (1.6 mol) of N-methyl-amino-2-ethanol were added and the mixture was stirred and heated to 160° C. The mixture was reacted for 8 hours and then allowed to cool to room temperature (25° C.). 150 ml of cold water were then added. The precipitate was filtered off, washed with acidified water and then with water, and then oven-dried at 60° C. under reduced pressure.

10.3 g of a violet-brown powder were obtained (90.9% yield).

Characterization

¹H NMR (CDCl₃, 400 MHz) δ: 8.75-8.35 (4H), 7.65-7.15 (7H), 3.93-3.91 (2H), 3.44-3.40 (2H), 3.02-2.98 (3H).

### Step 3: Preparation of naphthylisoguinolinone-4-N-methylaminoethane acrylate

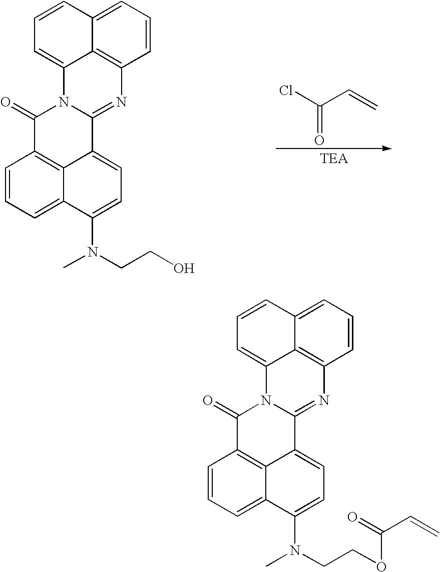

4.0 g (10.2 mmol) of naphthylisoquinolinone-4-N-methylaminoethanol were placed in a round-bottomed flask under an inert atmosphere of argon; 125 ml of dichloromethane (DCM) were added and stirring was continued until dissolution was complete. 4.6 g of triethylamine (45.9 mmol) were added with stirring. 1.1 g of acryloyl chloride (12.2 mmol) were diluted in 10 ml of DCM, and this solution was added dropwise to the reaction medium at 38° C. The mixture was heated to 40° C. with continued stirring, and was left to react for 12 hours. 20 ml of cold water were then added. The mixture was extracted-with 100 ml of aqueous sodium bicarbonate solution and then washed with 200 ml of water. The product was recovered, and was oven-dried at 35° C. under reduced pressure.

3.7 g of a violet-red powder were obtained (80% yield).

Characterization

1H NMR (CDCl₃, 400 MHz) δ:8.80-8.30 (4H), 7.66-7.21 (7H), 6.39-6.31 (1H), 6.12-6.03 (1H), 5.85-5.81 (1H), 4.49-4.46 (2H), 3.64-3.56 (2H), 3.09-3.05 (3H).

### EXAMPLE 2

33 g of isododecane were placed in a 500 ml reactor and then heated to 90° C. 2 g of the monomer prepared according to Example 1 were dissolved in 10 ml of toluene, and 40 g of isobornyl acrylate, 28 g of isobutyl methacrylate, 37 g of isododecane, and 0.6 g of initiator 2,5-bis(2-ethylhexanoylperoxy)-2,5-dimethylhexane (Trigonox® 141 from Akzo Nobel) were then added. This mixture was added over 1 hour in the reactor at 90° C. The whole was maintained at 90° C. for 1 hour 30 minutes.

30 g of 2-ethylhexyl acrylate, 30 g of isododecane, and 0.4 g of 2,5-bis(2-ethyl-hexanoylperoxy)-2,5-dimethylhexane were then introduced into the above mixture, at 90° C. and over 30 minutes. The mixture was maintained at 90° C. for 4 hours and was then cooled to 25° C.

After replacing the toluene with isododecane, a solution comprising 50% of polymer solids in isododecane was obtained. This polymer comprised a first block of isobornyl acrylate, isobutyl methacrylate, and optical-brightening monomer according to the present disclosure, a 2-ethylhexyl acrylate second block, and an intermediate segment.

### EXAMPLE 3

Optical Brightening Monomer

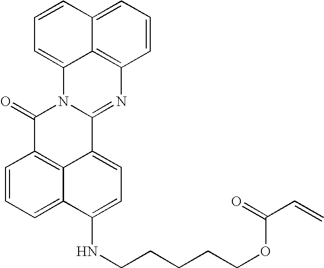

50 g of ethyl acetate were placed in a 500 ml reactor and then heated at 78° C. for 1 hour. 29.5 g of methyl methacrylate, 5 g of acrylic acid, 0.5 g of above monomer dissolved in 15 g of THF, and 0.3 g of 2,5-bis(2-ethylhexanoylperoxy)-2,5-dimethylhexane (Trigono)® 141 from Akzo Nobel) were then added, at 78° C. and over 40 minutes. The mixture was maintained at 78° C. for 1 hour. 15 g of methyl acrylate and 0.2 g of 2,5-bis(2-ethylhexanoylperoxy)-2,5-dimethylhexane were then added at 78° C. and over 30 minutes. The mixture was maintained at 78° C. for 5 hours and then diluted with 75 g of butyl acetate. The ethyl acetate and the THF were distilled off under reduced pressure. 50 g of butyl acetate were then added and 50 g of butyl acetate were distilled off.

A solution comprising 31.2% of polymer solids in butyl acetate was obtained. This polymer comprised a first block of methyl methacrylate, acrylic acid, and monomer according to the present disclosure, a methyl acrylate second block, and an intermediate segment.

### EXAMPLE 4

An anhydrous foundation comprising the following (weight %) was prepared:

| | |
|---|---|
| polyethylene wax | 12% |
| volatile silicone oils | 25% |
| phenyl trimethicone | 20% |
| polymethyl methacrylate microspheres | 12% |
| polymer of Example 2 | 6% |
| isododecane | qs |
| | 100% |

Preparation:

The waxes were melted and, when the solution was clear, the phenyl trimethicone and the silicone oils were added with stirring. The microspheres, the isododecane, and the polymer were then added. The mixture was homogenized for 15 minutes and the resulting composition was then cast and allowed to cool.

An anhydrous foundation was obtained.

### EXAMPLE 5

A nail varnish was prepared, comprising:
20% by weight of polymer according to Example 3
qs 100% organic solvents (butyl acetate and ethyl acetate).

### EXAMPLE 6

A stick of lipstick was prepared, comprising:

| | |
|---|---|
| polyethylene wax | 15% |
| polymer of Example 2 | 10% |
| | AM |
| hydrogenated polyisobutene (Parléam from Nippon Oil | 25% |
| Fats) | |
| Pigments | 10% |
| Isododecane | qs |
| | 100% |

1. A block polymer comprising at least one first block and at least one second block that are mutually incompatible, wherein the at least one first block and the at least one second block are linked together via an intermediate segment comprising at least one constituent monomer of the at least one first block and at least one constituent monomer of the at least one second block, and wherein at least one of the blocks comprises at least one monomer of formula (I): 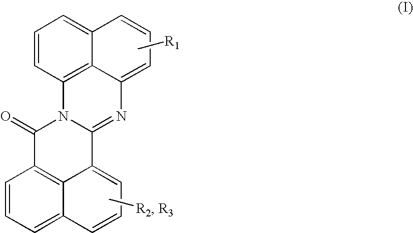
wherein:
R₁ is chosen from hydrogen; halogen atoms; linear, branched and cyclic, saturated and unsaturated carbon-based radicals comprising from 1 to 12 carbon atoms, optionally substituted with at least one substituent chosen from ═O, OH, and NH₂, and optionally interrupted with at least one heteroatom chosen from O, N, P, Si, and S; groups of formula NRR′, wherein R and R′, which are identical or different, are chosen from hydrogen and linear, cyclic and branched, saturated hydrocarbon-based radicals comprising from 1 to 6 carbon atoms; and
R₂ and R₃, which are present on the same ring or on a different ring, and which are identical or-different, are chosen from hydrogen, halogen atoms, and groups of formula (II), with the proviso that at least one of the radicals R₂ and R₃ is chosen from groups of formula (II ):
—X-G-P (II)
wherein:
X is chosen from the entities —O—, —S—, —SO—, —SO₂—, —NH—, and —NR″—, wherein R″ is chosen from linear, branched and cyclic, saturated and unsaturated carbon-based radicals comprising from 1 to 30 carbon atoms, optionally substituted with at least one group chosen from ═O, OH, NH₂, and halogen atoms, and optionally interrupted with at least one heteroatom chosen from O, N, P, Si, and S;
G is chosen from linear, branched and cyclic, saturated and unsaturated divalent carbon-based radicals comprising from 1 to 32 carbon atoms, optionally substituted with at least one group chosen from ═O, OH, NH₂, and halogen atoms, and optionally interrupted with at least one heteroatom chosen from O, N, P, Si, and S;
P is a polymerizable group chosen from those of formulae (IIIa), (IIIb), and (IIIc): 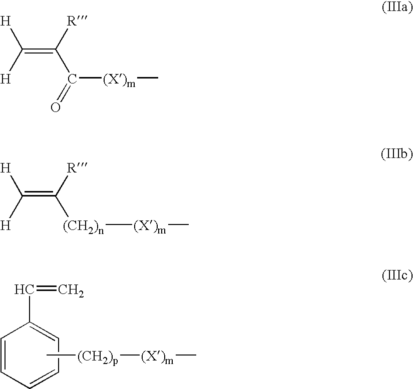
wherein:
R′″ is chosen from hydrogen and linear and branched, saturated C₁-C₆ hydrocarbon-based radicals,
X′ is chosen from O, NH, and NR″″, wherein R″″ is chosen from C₁-C₆ alkyl, C₆-C₁₀ aryl, (C₆-C₁₀)aryl(C₁-C₆)alkyl, and (C₁-C₆)alkyl(C₆-C₁₀)aryl radicals, wherein the alkyl and aryl groups are optionally substituted with at least one substituent chosen from OH, halogen atoms, and C₁-C₆ alkoxy and C₆-C₁₀ aryloxy radicals;
m is equal to 0 or 1;
n is equal to 0 or 1; and
p is equal to 0, 1 or 2.

2. The polymer according to claim 1, wherein R₁ is hydrogen.

3. The polymer according to claim 1, wherein R₂ is hydrogen and R₃ is a group of formula (II).

4. The polymer according to claim 1, wherein, in the group of formula (II), X is chosen from —O— and —NR″—, wherein R″ is chosen from linear, branched and cyclic, saturated and unsaturated hydrocarbon-based radicals, optionally comprising a hydrocarbon-based ring that is itself saturated or unsaturated, said hydrocarbon-based radicals comprising from 2 to 18 carbon atoms, and said hydrocarbon-based radicals being optionally substituted with at least one substituent chosen from ═O, OH, NH₂, and halogen atoms, and being optionally interrupted with at least one heteroatom chosen from O, N, P, Si, and S.

5. The polymer according to claim 1, wherein R″ is chosen from ethyl, n-propyl, isopropyl, n-butyl, isobutyl, tert-butyl, pentyl, hexyl, cyclohexyl, octyl, cyclooctyl, decyl, cyclodecyl, dodecyl, cyclodecyl, phenyl, and benzyl radicals.

6. The polymer according to claim 1, wherein G is chosen from linear, branched and cyclic, saturated and unsaturated divalent hydrocarbon-based radicals, optionally comprising a hydrocarbon-based ring that is itself saturated or unsaturated, said hydrocarbon-based radicals comprising2 to 18 carbon atoms, and said hydrocarbon-based radicals being optionally substituted with at least one substituent chosen from ═O, OH, NH₂, and halogen atoms, and being optionally interrupted with at least one heteroatom chosen from O, N, P, and Si.

7. The polymer according to claim 6, wherein G is chosen from linear and branched, saturated divalent hydrocarbon-based radicals optionally comprising a saturated hydrocarbon-based ring, said hydrocarbon-based radicals comprising 2 to 18 carbon atoms.

8. The polymer according to claim 6, wherein G is chosen from ethylene, n-propylene, isopropylene, n-butylene, isobutylene, pentylene, hexylene, cyclohexylene, heptylene, octylene, cyclooctylene, decylene, cyclodecylene, cyclohexyldimethylene, dodecylene, and cyclododecylene radicals.

9. The polymer according to claim 1, wherein the polymerizable group P is chosen from one of the following formulae: 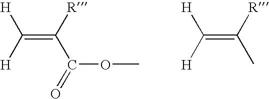
wherein R′″ is chosen from hydrogen and methyl.

10. The polymer according to claim 1, wherein the at least one monomer of formula (I) is chosen from those of the following formulae, wherein R is H or methyl:

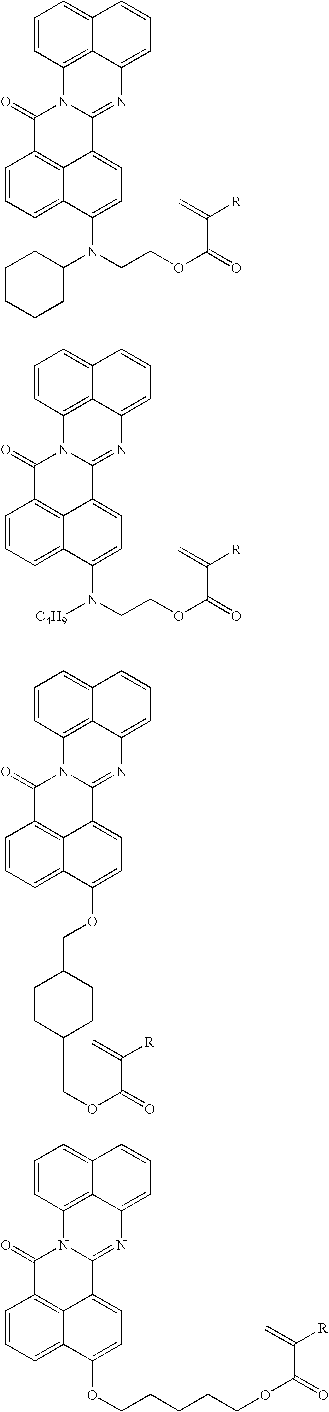

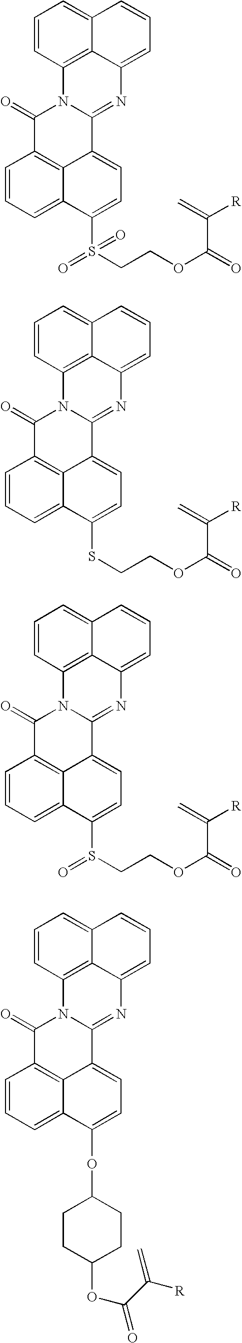

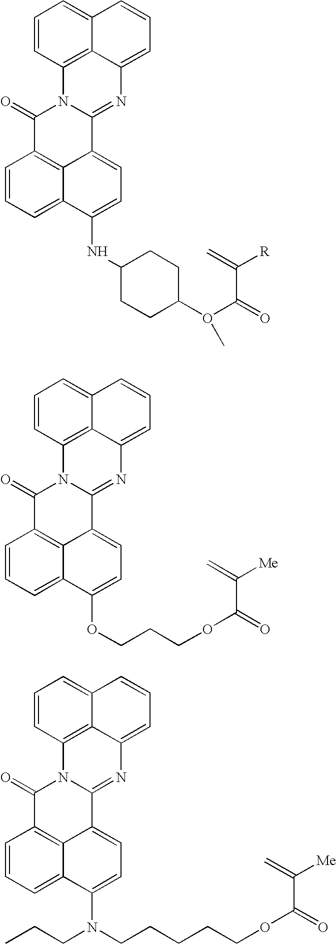

11. The polymer according to claim 1, further comprising at least one additional monomer chosen from the following monomers:
(i) ethylenic hydrocarbons comprising from 2 to 10 carbons;
(ii) the (meth)acrylates of formulae: 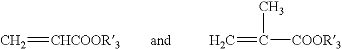
wherein R′₃ is chosen from:
linear and branched alkyl groups comprising from 1 to 18 carbon atoms, optionally intercalated with at least-one heteroatom chosen from O, N, S, and P, and optionally substituted with at least one substituent chosen from hydroxyl groups, halogen atoms, and groups of formula Si(R₄R₅), wherein R₄ and R₅, which may be identical or different, are chosen from C₁ to C₆ alkyl groups and phenyl groups;
C₃ to C₁₂ cycloalkyl groups;
C₃ to C₂₀ aryl groups;
C₄ to C₃₀ aralkyl groups (C₁ to C₈ alkyl groups);
4- to 12-membered heterocyclic groups comprising at least one heteroatom chosen from O, N, and S, the ring being aromatic or non-aromatic;
heterocycloalkyl groups (C₁ to C₄ alkyl groups);
wherein the cycloalkyl, aryl, aralkyl, heterocyclic, and heterocycloalkyl groups optionally substituted with at least one substituent chosen from hydroxyl groups, halogen atoms, and linear and branched C₁-C₄ alkyl groups optionally intercalated with at least one heteroatom chosen from O, N, S, and P, the alkyl groups also possibly being optionally substituted with at least one substituent chosen from hydroxyl groups, halogen atoms, and groups of formula Si(R4R₅), wherein R₄ and R₅, which may be identical or different, are chosen from C₁ to C₆ alkyl groups and phenyl groups; and
groups of formula —(C₂H₄O)_{q}—R₁₃, wherein q ranges from 5 to 150 and R₁₃ is chosen from hydrogen and C₁ to C₃₀ alkyl groups;
(iii) the (meth)acrylamides of formula: 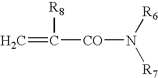
wherein:
R₈ is chosen from hydrogen and methyl; and
R₆ and R₇, which may be identical or different, are chosen from:
hydrogen;
linear and branched alkyl groups comprising from 1 to 18 carbon atoms, optionally intercalated with at least one heteroatom chosen from O, N, S, and P, and optionally substituted with at least one substituent chosen from hydroxyl groups, halogen atoms, and groups of formula Si(R₄R₅), wherein R₄ and R₅, which may be identical or different, are chosen from C₁ to C₆ alkyl groups and phenyl groups;
C₃ to C₁₂ cycloalkyl groups;
C₃ to C₂₀ aryl groups;
C₄ to C₃₀ aralkyl groups (C₁ to C₈ alkyl groups);
4- to 12-membered heterocyclic groups comprising at least one heteroatom chosen from O, N, and S, the ring being aromatic or non-aromatic; and
heterocycloalkyl groups (C₁ to C₄ alkyl groups);
wherein the cycloalkyl, aryl, aralkyl, heterocyclic, and heterocycloalkyl groups are optionally substituted with at least one substituent chosen from hydroxyl groups, halogen atoms, and linear and branched C₁-C₄ alkyl groups optionally. intercalated with at least one heteroatom chosen from O, N, S, and P, the alkyl groups optionally substituted with at least one substituent chosen from hydroxyl groups, halogen atoms, and groups of formula Si(R₄R₅), wherein R₄ and R₅, which may be identical or different, are chosen from C₁ to C₆ alkyl groups and phenyl groups;
(iv) the vinyl compounds of formulae:
CH₂═CH—R₉, CH₂═CH—CH₂—R₉, and CH₂═C(CH₃)—CH₂—R₉
wherein R₉ is chosen from hydroxyl groups; halogen atoms; NH₂; groups of formula OR₁₀, wherein R₁₀ is chosen from phenyl groups and C₁ to C₁₂ alkyl groups; acetamide (NHCOCH₃); groups of formula OCOR₁₁, wherein R₁₁ is chosen from linear and branched alkyl groups comprising from 2 to 12 carbon atoms; and groups chosen from:
linear and branched alkyl groups comprising from 1 to 18 carbon atoms, optionally intercalated with at least one heteroatom chosen from O, N, S, and P, and optionally substituted with at least one substituent chosen from hydroxyl groups, halogen atoms, and groups of formula Si(R₄R₅), wherein R₄ and R₅, which may be identical or different, are chosen from C₁ to C₆ alkyl groups and phenyl groups;
C₃ to C₁₂ cycloalkyl groups;
C₃ to C₂₀ aryl groups;
C₄ to C₃₀ aralkyl groups (C₁ to C₈ alkyl groups);
4- to 12-membered heterocyclic groups comprising at least one heteroatom chosen from O, N, and S, the ring being aromatic or non-aromatic; and
heterocycloalkyl groups (C₁ to C₄ alkyl groups);
wherein the cycloalkyl, aryl, aralkyl, heterocyclic, and heterocycloalkyl group areoptionally substituted with at least one substituent chosen from hydroxyl groups, halogen atoms, and linear and branched C₁ to C₄ alkyl groups optionally intercalated with at least one heteroatom chosen from O, N, S, and P, and optionally substituted with at least one substituent chosen from hydroxyl groups, halogen atoms, and groups of formula Si(R₄R₅), wherein R₄ and R₅, which may be identical or different, are chosen from C₁ to C₆ alkyl groups and phenyl groups;
(v) (meth)acrylic, (meth)acrylamide, and vinyl monomers comprising a fluoro or perfluoro group;
(vi) silicone-based (meth)acrylic, (meth)acrylamide, and vinyl monomers;
(vii) ethylenically unsaturated monomers comprising at least one functional group chosen from carboxylic, phosphoric, and sulfonic acid, and anhydride, functional groups; and
(viii) ethylenically unsaturated monomers comprising at least one tertiary amine functional group.

12. The polymer according to claim 1, further comprising at least one additional monomer chosen, from those with an optical effect of formula (A), (B) and (C): 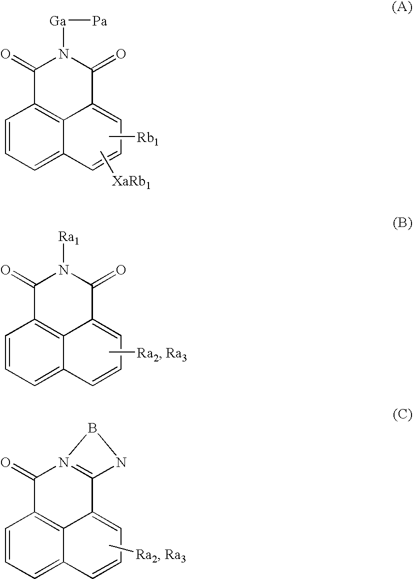
wherein:
Ra₁ is chosen from linear, branched and cyclic, saturated and unsaturated carbon-based radicals comprising from 1 to 32 carbon atoms, optionally substituted with at least one substituent chosen from ═O, OH, NH₂, and halogen atoms, and/or optionally interrupted with at least one heteroatom chosen from O, N, P, Si, and S;
Rb₁ is chosen from hydrogen; halogen; linear, branched and/or cyclic, saturated and/or unsaturated carbon-based radicals comprising from 1 to 12 carbon atoms, optionally substituted with at least one substituent chosen from ═O, OH, and NH₂, and/or optionally interrupted with at least one heteroatom chosen from O, N, P, Si, and S; groups of formula NRR′, wherein R and R′, which are identical or different, are chosen from hydrogen and linear, cyclic and branched, saturated C₁ to C₆ hydrocarbon-based radicals;
Ra₂ and Ra₃, which are present on the same ring or on a different ring, and which are identical or different, are chosen from hydrogen, halogen atoms, and groups of formula (III), with the proviso that at least one of the radicals Ra₂ and Ra₃ is chosen from groups of formula (III):
—Xa-Ga-Pa (III)
wherein:
Xa is chosen from the groups —O—, —S—, —SO—, —SO₂—, —NH—, and —NR″—, wherein R″ is chosen from linear, branched and/or cyclic, saturated and/or unsaturated carbon-based radicals comprising from 1 to 30 carbon atoms, optionally substituted with at least one substituent chosen from ═O, OH, NH₂, and halogen atoms, and/or optionally interrupted with at least one heteroatom chosen from O, N, P, Si, and S;
Ga is chosen from linear, branched and/or cyclic, saturated and/or unsaturated divalent carbon-based radicals comprising 1 to 32 carbon atoms, optionally substituted with at least one substituent chosen from ═O, OH, NH₂, and halogen atoms, and/or optionally interrupted with at least one heteroatom chosen from O, N, P, Si, and S;
Pa is a polymerizable group chosen from those of formulae (IIIa), (IIIb), and (IIIc): 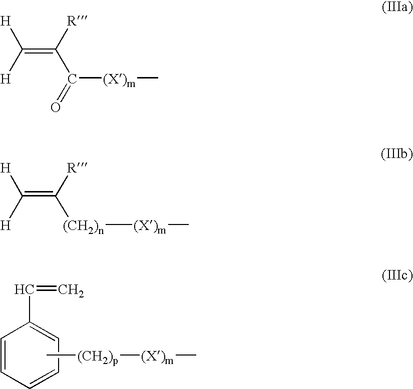
wherein:
R′″ is chosen from hydrogen and linear and branched, saturated C₁-C₆ hydrocarbon-based radicals,
X′ is chosen from O, NH, and NR″″, wherein R″″ is chosen from C₁-C₆ alkyl, C₆-C₁₀aryl, (C₆-C₁₀)aryl(C₁-C₆)alkyl, and (C₁-C₆)alkyl(C₆-C₁₀)aryl radicals, wherein the alkyl and/or aryl groups are optionally substituted with at least one substituent chosen from OH, halogen, and C₁-C₆ alkoxy and C₆₋₁₀ aryloxy-radicals;
m is equal to 0 or 1;
n is equal to 0 or 1; and
p is equal to 0, 1 or 2; and
B is chosen from one of the following divalent aromatic groups of formulae (IVa) to (IVd): 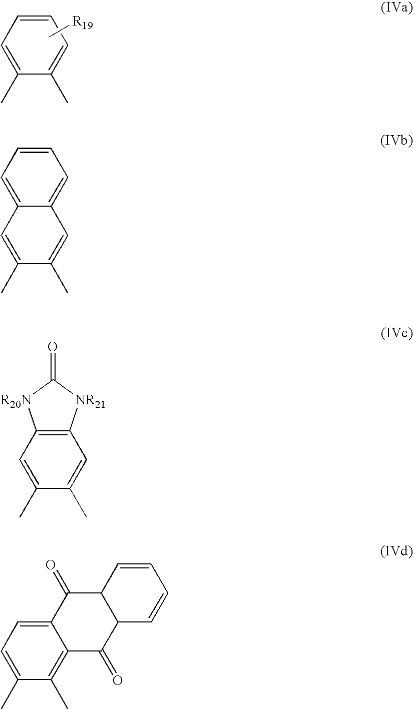
wherein:
R₁₉ is chosen from linear, branched and/or cyclic, saturated and/or unsaturated carbon-based radicals comprising from 1 to 32 carbon atoms, optionally substituted with at least one substituent chosen from ═O, OH, NH₂, and halogen atoms; and
R₂₀ and R₂₁, which may be identical or different, are chosen from hydrogen, linear and branched C₁-C₈ alkyl radicals and cyclopentyl, cyclohexyl, cyclooctyl, cyclodecyl, cyclododecyl, benzyl, naphthyl, and phenyl radicals.

13. The polymer according to claim 1, wherein the at least one monomer of formula (I) is present in an amount ranging from 0.01% to 100% by weight, relative to the weight of the block comprising it.

14. The polymer according to claim 1, wherein the at least one monomer of formula (I) is present in an amount ranging from 0.01% to 70% by weight, relative to the total weight of the polymer.

15. A composition, comprising, in a physiologically acceptable medium, at least one block polymer comprising at least one first block and at least one second block that are mutually incompatible, wherein the at least one first block and the at least one second block are linked together via an intermediate segment comprising at least one constituent monomer of the at least one first block and at least one constituent monomer of the at least one second block, and wherein at least one of the blocks comprises at least one monomer of formula (I): 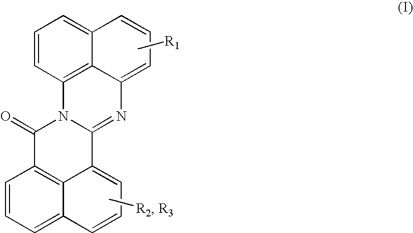
wherein:
R₁ is chosen from hydrogen; halogen atoms; linear, branched and cyclic, saturated and unsaturated carbon-based radicals comprising from 1 to 12 carbon atoms, optionally substituted with at least one substituent chosen from ═O, OH, and NH₂, and optionally interrupted with at least one heteroatom chosen from O, N, P, Si, and S; groups of formula NRR′, wherein R and R′, which may be identical or different, are chosen from hydrogen and linear, cyclic and branched, saturated hydrocarbon-based radicals comprising from 1 to 6 carbon atoms; and
R₂ and R₃, which are present on the same ring or on a different ring, and which are identical or different, are chosen from hydrogen, halogen atoms, and groups of formula (II), with the proviso that at least one of the radicals R₂ and R₃ is chosen from groups of formula (II):
—X-G-P (II)
wherein:
X is chosen from the entities —O—, —S—, —SO—, —SO₂—, —NH—, and —NR″—, wherein R″ is chosen from linear, branched and cyclic, saturated and unsaturated carbon-based radicals comprising from 1 to 30 carbon atoms, optionally substituted with at least one group chosen from ═O, OH, NH₂, and halogen atoms, and optionally interrupted with at least one heteroatom chosen from O, N, P, Si, and S;
G is chosen from linear, branched and cyclic, saturated and unsaturated divalent carbon-based radicals comprising from 1 to 32 carbon atoms, optionally substituted with at least one group chosen from ═O, OH, NH₂, and halogen atoms, and optionally interrupted with at least one heteroatom chosen from O, N, P, Si, and S;
P is a polymerizable group chosen from those of formulae (IIIa), (IIIb), and (IIIc): 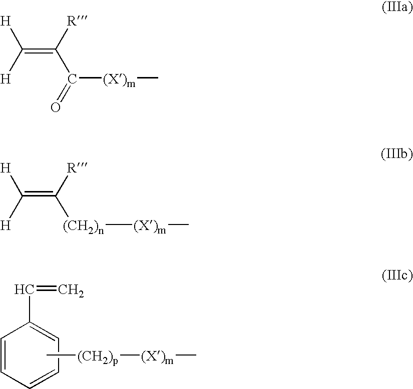
wherein:
R′″ is chosen from hydrogen and linear and branched, saturated C₁-C₆ hydrocarbon-based radicals,
X′ is chosen from O, NH, and NR″″, wherein R″″ is chosen from C₁-C₆ alkyl, C₆-C₁₀ aryl, (C₆-C₁₀)aryl(C₁-C₆)alkyl, and (C₁-C₆)alkyl(C₆-C₁₀)aryl radicals, wherein the alkyl and aryl groups are optionally substituted with at least one substituent chosen from OH, halogen atoms, and C₁-C₆ alkoxy and C₆-C₁₀ aryloxy radicals;
m is equal to 0 or 1;
n is equal to 0 or 1; and
p is equal to 0, 1 or 2.

16. The composition according to claim 15, wherein the at least one block polymer is present in an amount ranging from 0.01 % to 75% by weight, relative to the total weight of the composition.

17. The composition according to claim 15, wherein the physiologically acceptable medium comprises a hydrophilic medium comprising water or a mixture of water/hydrophilic organic solvent(s) and optionally comprising a fatty phase.

18. The composition according to claim 17, wherein the fatty phase comprises at least one compound chosen from waxes, pasty fatty substances, gums, lipophilic organic solvents and oils, and/or mixtures thereof.

19. The composition according to claim 15, further comprising a particulate phase comprising at least one compound chosen from at least one pigment, at least one nacre, and at least one filler.

20. The composition according to claim 15, further comprising at least one dyestuff chosen from water-soluble dyes and/or liposoluble dyes.

21. The composition according to claim 15, further comprising at least one additional polymer.

22. The composition according to claim 21, wherein the at least one additional polymer is a film forming polymer.

23. The composition according to claim 15, further comprising at least one ingredient chosen from vitamins, thickeners, gelling agents, trace elements, softeners, sequestering agents, fragrances, acidifying agents, basifying agents, preserving agents, sunscreens, surfactants, antioxidants, agents for preventing hair loss, antidandruff agents, propellants, ceramides, and mixtures thereof.

24. The composition according to claim 15, wherein the composition is in a form chosen from a suspension,.a dispersion, an optionally thickened and/or gelled oily solution, an oil-in-water emulsion, a water-in-oil emulsion, a multiple emulsion, a gel, a mousse, an oily or emulsified gel, a dispersion of vesicles, a two-phase or multiphase lotion, a spray, a free powder, a compact powder, a cast powder, an anhydrous paste, a lotion, a cream, a salve, a soft paste, an ointment, a cast solid, a moulded solid, and a compacted solid.

25. The composition according to claim 15, wherein the composition is in a form chosen from a care and/or makeup product for bodily or facial skin, the lips, the nails, the eyelashes, the eyebrows and/or the hair, an antisun product, a self-tanning product, and a hair product for caring for, treating, shaping, making up, and/or coloring the hair.

26. The composition according to claim 15, wherein the composition is in a form of a makeup composition.

27. The composition according to claim 25, wherein the makeup composition is chosen from a foundation; a makeup rouge; an eyeshadow; a lipstick; a lipcare product; a concealer product; a blusher; a mascara; an eyeliner; an eyebrow makeup product; a lip pencil; an eye pencil; a nail product; a body makeup product; a protective composition for the skin of the face, the neck, the hands, or the body; a moisturizing or treating composition; an antisun composition; an artificial tanning composition; and a hair product.

28. A cosmetic treatment process for making up and/or caring for keratin materials, comprising:
applying to the keratin materials a composition, comprising, in a physiologically acceptable medium, at least one block polymer comprising at least one first block and at least one second block that are mutually incompatible, wherein the at least one first block and the at least one second block are linked together via an intermediate segment comprising at least one constituent monomer of the at least one first block and at least one constituent monomer of the at least one second block, and wherein at least one of the blocks comprises at least one monomer of formula (I): 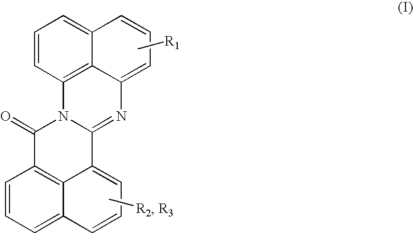
wherein:
R₁ is chosen from hydrogen; halogen atoms; linear, branched and cyclic, saturated and unsaturated carbon-based radicals comprising from 1 to 12 carbon atoms, optionally substituted with at least one substituent chosen from ═O, OH, and NH₂ and optionally interrupted with at least one heteroatom chosen from O, N, P, Si, and S; groups of formula NRR′, wherein R and R′, which are identical or different, are chosen from hydrogen and linear, cyclic and branched, saturated hydrocarbon-based radicals comprising from 1 to 6 carbon atoms; and
R₂ and R₃, which are present on the same ring or on a different ring, and which are identical or different, are chosen from hydrogen, halogen atoms, and groups of formula (II), with the proviso that at least one of the radicals R₂ and R₃ is chosen from groups of formula (II):
—X-G-P (II)
wherein:
X is chosen from the entities —O—, —S—, —SO—, —SO₂—, —NH—, and —NR″—, wherein R″ is chosen from linear, branched and cyclic, saturated and unsaturated carbon-based radicals comprising from 1 to 30 carbon atoms, optionally substituted with at least one group chosen from ═O, OH, NH₂, and halogen atoms, and optionally interrupted with at least one heteroatom chosen from O, N, P, Si, and S;
G is chosen from linear, branched and cyclic, saturated and unsaturated divalent carbon-based radicals comprising from 1 to 32 carbon atoms, optionally substituted with at least one group chosen from ═O, OH, NH₂, and halogen atoms, and optionally interrupted with at least one heteroatom chosen from O, N, P, Si, and S;
P is a polymerizable group chosen from those of formulae (IIIa), (IIIb), and (IIIc): 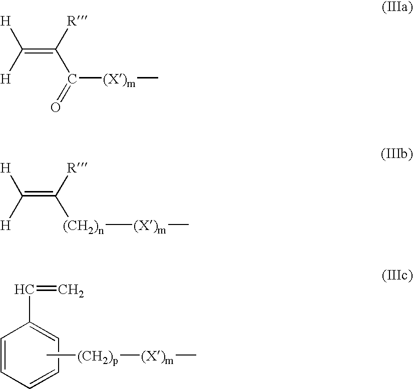
wherein:
R′″ is chosen from hydrogen and linear and branched, saturated C₁-C₆ hydrocarbon-based radicals,
X′ is chosen from O, NH, and NR″″, wherein R″″ is chosen from C₁-C₆ alkyl, C₆-C₁₀ aryl, (C₆-C₁₀)aryl(C₁-C₆)alkyl, and (C₁-C₆)alkyl(C₆-C₁₀)aryl radicals, wherein the alkyl and aryl groups are optionally substituted with at least one substituent chosen from OH, halogen atoms, and C₁-C₆ alkoxy and C₆-C₁₀ aryloxy radicals;
m is equal to 0 or 1;
n is equal to 0 or 1; and
p is equal to 0, 1 or 2.

29. A nail varnish composition comprising at least one organic solvent, at least one film-forming polymer, optionally at least one pigment, optionally at least one dye, and at least one block polymer comprising at least one first block and at least one second block that are mutually incompatible, wherein the at least one first block and the at least one second block are linked together via an intermediate segment comprising at least one constituent monomer of the at least one first block and at least one constituent monomer of the at least one second block, and wherein at least one of the blocks comprises at least one monomer of formula (I): 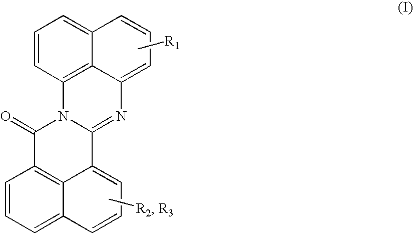
wherein:
R₁ is chosen from hydrogen; halogen atoms; linear, branched and/cyclic, saturated and unsaturated carbon-based radicals comprising from 1 to 12 carbon atoms, optionally substituted with at least one substituent chosen from ═O, OH, and NH₂ and optionally interrupted with at least one heteroatom chosen from O, N, P, Si, and S; groups of formula NRR′, wherein R and R′, which are identical or different, are chosen from hydrogen and linear, cyclic and branched, saturated hydrocarbon-based radicals comprising from 1 to 6 carbon atoms; and
R₂ and R₃, which are present on the same ring or on a different ring, and which are identical or different, are chosen from hydrogen, halogen atoms, and groups of formula (II), with the proviso that at least one of the radicals R₂ and R₃ is chosen from groups of formula (II):
—X-G-P (II)
wherein:
X is chosen from the entities —O—, —S—, —SO—, —SO₂—, —NH—, and —NR″—, wherein R″ is chosen from linear, branched and cyclic, saturated and unsaturated carbon-based radicals comprising from 1 to 30 carbon atoms, optionally substituted with at least one group chosen from ═O, OH, NH₂, and halogen atoms, and optionally interrupted with at least one heteroatom chosen from O, N, P, Si, and S;
G is chosen from linear, branched and cyclic, saturated and unsaturated divalent carbon-based radicals comprising from 1 to 32 carbon atoms, optionally substituted with at least one group chosen from ═O, OH, NH₂, and halogen atoms, and optionally interrupted with at least one heteroatom chosen from O, N, P, Si, and S;
P is a polymerizable group chosen from those of formulae (IIIa), (IIIb), and (IIIc): 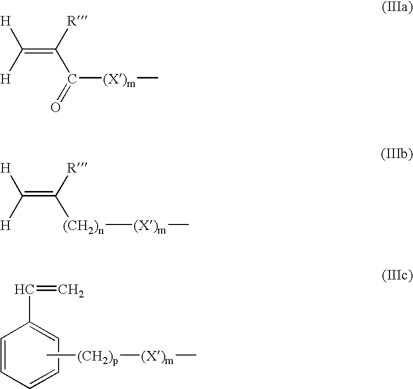
wherein:
R′″ is chosen from hydrogen and linear and branched, saturated C₁-C₆ hydrocarbon-based radicals,
X′ is chosen from O, NH, and NR″″, wherein R″″ is chosen from C₁-C₆ alkyl, C₆-C₁₀ aryl, (C₆-C₁₀)aryl(C₁-C₆)alkyl, and (C₁-C₆)alkyl(C₆-C₁₀)aryl radicals, wherein the alkyl and aryl groups are optionally substituted with at least one substituent chosen from OH, halogen atoms, and C₁-C₆ alkoxy and C₆-C₁₀ aryloxy radicals;
m is equal to 0 or 1;
n is equal to 0 or 1; and
p is equal to 0, 1 or 2.

30. A foundation composition comprising at least one oil in a fatty phase, at least one pigment, optionally an aqueous phase, and at least one block polymer comprising at least one first block and at least one second block that are mutually incompatible, wherein the at least one first block and the at least one second block are linked together via an intermediate segment comprising at least one constituent monomer of the at least one first block and at least one constituent monomer of the at least one second block, and wherein at least one of the blocks comprises at least one monomer of formula (I): 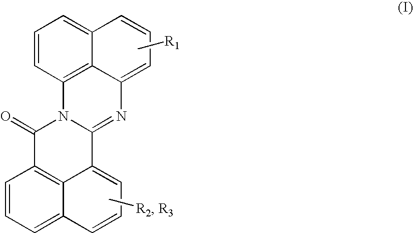
wherein:
R₁ is chosen from hydrogen; halogen atoms; linear, branched and cyclic, saturated and unsaturated carbon-based radicals comprising from 1 to 12 carbon atoms, optionally substituted with at least one substituent chosen from ═O, OH, and NH₂ and optionally interrupted with at least one heteroatom chosen from O, N, P, Si, and S; groups of formula NRR′, wherein R and R′, which are identical or different, are chosen from hydrogen and linear, cyclic and branched, saturated hydrocarbon-based radicals comprising from 1 to 6 carbon atoms; and
R₂ and R₃, which are present on the same ring or on a different ring, and which are identical or different, are chosen from hydrogen, halogen atoms, and groups of formula (II), with the proviso that at least one of the radicals R₂ and R₃ is chosen from groups of formula (11):
—X-G-P (II)
wherein:
X is chosen from the entities —O—, —S—, —SO—, —SO₂—, —NH—, and —NR″—, wherein R″ is chosen from linear, branched and/or cyclic, saturated and/or unsaturated carbon-based radicals comprising from 1 to 30 carbon atoms, optionally substituted with at least one group chosen from ═O, OH, NH₂, and halogen atoms, and optionally interrupted with at least one heteroatom chosen from O, N, P, Si, and S;
G is chosen from linear, branched and cyclic, saturated and unsaturated divalent carbon-based radicals comprising from 1 to 32 carbon atoms, optionally substituted with at least one group chosen from ═O, OH, NH₂, and halogen atoms, and optionally interrupted with at least one heteroatom chosen from O, N, P, Si, and S;
P is a polymerizable group chosen from those of formulae (IIIa), (IIIb), and (IIIc): 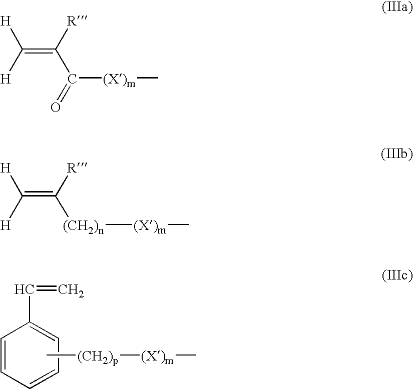
wherein:
R′″ is chosen from hydrogen and linear and branched, saturated C₁-C₆ hydrocarbon-based radicals,
X′ is chosen from O, NH, and NR″″, wherein R″″ is chosen from C₁-C₆ alkyl, C₆-C₁₀ aryl, (C₆-C₁₀)aryl(C₁-C₆)alkyl, and (C₁-C₆)alkyl(C₆-C₁₀)aryl radicals, wherein the alkyl and aryl groups may be substituted with at least one substituent chosen from OH, halogen atoms, and C₁-C₆ alkoxy and C₆-C₁₀ aryloxy radicals;
m is equal to 0 or 1;
n is equal to 0 or 1; and
p is equal to 0, 1 or 2.

31. An anti-ageing and/or anti-wrinkle composition comprising at least one block polymer comprising at least one first block and at least one second block that are mutually incompatible, wherein the at least one first block and the at least one second block are linked together via an intermediate segment comprising at least one constituent monomer of the at least one first block and at least one constituent monomer of the at least one second block, and wherein at least one of the blocks comprises at least one monomer of formula (I): 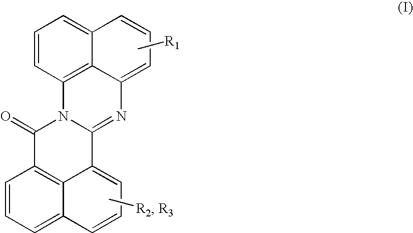
wherein:
R₁ is chosen from hydrogen; halogen atoms; linear, branched and cyclic, saturated and unsaturated carbon-based radicals comprising from 1 to 12 carbon atoms, optionally substituted with at least one substituent chosen from ═O, OH, and NH₂ and optionally interrupted with at least one heteroatom chosen from O, N, P, Si, and S; groups of formula NRR′, wherein R and R′, which are identical or different, are chosen from hydrogen and linear, cyclic and branched, saturated hydrocarbon-based radicals comprising from 1 to 6 carbon atoms; and
R₂ and R₃, which are present on the same ring or on a different ring, and which are identical or different, are chosen from hydrogen, halogen atoms, and groups of formula (II), with the proviso that at least one of the radicals R₂ and R₃ is chosen from groups of formula (II):
—X-G-P (II)
wherein:
X is chosen from the entities —O—, —S—, —SO—, —SO₂—, —NH—, and —NR″—, wherein R″ is chosen from linear, branched and cyclic, saturated and unsaturated carbon-based radicals comprising from 1 to 30 carbon atoms, optionally substituted with at least one group chosen from ═O, OH, NH₂, and halogen atoms, and optionally interrupted with at least one heteroatom chosen from O, N, P, Si, and S;
G is chosen from linear, branched and cyclic, saturated and unsaturated divalent carbon-based radicals comprising from 1 to 32 carbon atoms, optionally substituted with at least one group chosen from ═O, OH, NH₂, and halogen atoms, and optionally interrupted with at least one heteroatom chosen from O, N, P, Si, and S;
P is a polymerizable group chosen from those of formulae (IIIa), (IIIb), and (IIIc): 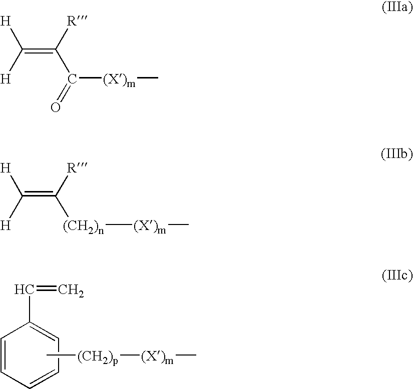
wherein:
R′″ is chosen from hydrogen and linear and branched, saturated C₁-C₆ hydrocarbon-based radicals,
X′ is chosen from O, NH, and NR″″, wherein R″″ is chosen from C₁-C₆ alkyl, C₆-C₁₀ aryl, (C₆-C₁₀)aryl(C₁-C₆)alkyl, and (C₁-C₆)alkyl(C₆-C₁₀)aryl radicals, wherein the alkyl and aryl groups may be substituted with at least one substituent chosen from OH, halogen atoms, and C₁-C₆ alkoxy and C₆-C₁₀ aryloxy radicals;
m is equal to 0 or 1;
n is equal to o or 1; and
p is equal to 0, 1 or 2.

32. A cosmetic process for treating wrinkled skin, comprising:
applying to the skin a cosmetic composition comprising, in a cosmetically acceptable medium, at least one block polymer comprising at least one first block and at least one second block that are mutually incompatible, wherein the at least one first block and the at least one second block are linked together via an intermediate segment comprising at least one constituent monomer of the at least one first block and at least one constituent monomer of the at least one second block, and wherein at least one of the blocks comprises at least one monomer of formula (I):
R₁ 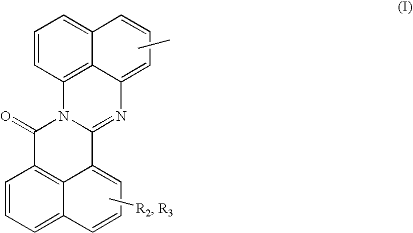
wherein:
R₁ is chosen from hydrogen; halogen atoms; linear, branched and cyclic, saturated and unsaturated carbon-based radicals comprising from 1 to 12 carbon atoms, optionally substituted with at least one substituent chosen from ═O, OH, and NH₂ and optionally interrupted with at least one heteroatom chosen from O, N, P, Si, and S; groups of formula NRR′, wherein R and R′, which are identical or different, are chosen from hydrogen and linear, cyclic and branched, saturated hydrocarbon-based radicals comprising from 1 to 6 carbon atoms; and
R₂ and R₃, which are present on the same ring or on a different ring, and which are identical or different, are chosen from hydrogen, halogen atoms, and groups of formula (II), with the proviso that at least one of the radicals R₂ and R₃ is chosen from groups of formula (II):
—X-G-P (II)
wherein:
X is chosen from the entities —O—, —S—, —SO—, —SO₂—, —NH—, and —NR″—, wherein R″ is chosen from linear, branched and cyclic, saturated and unsaturated carbon-based radicals comprising from 1 to 30 carbon atoms, optionally substituted with at least one group chosen from ═O, OH, NH₂, and halogen atoms, and optionally interrupted with at least one heteroatom chosen from O, N, P, Si, and S;
G is chosen from linear, branched and cyclic, saturated and unsaturated divalent carbon-based radicals comprising from 1 to 32 carbon atoms, optionally substituted with at least one group chosen from ═O, OH, NH₂, and halogen atoms, and optionally interrupted with at least one heteroatom chosen from O, N, P, Si, and S;
P is a polymerizable group chosen from those of formulae (IIIa), (IIIb), and (IIIc): 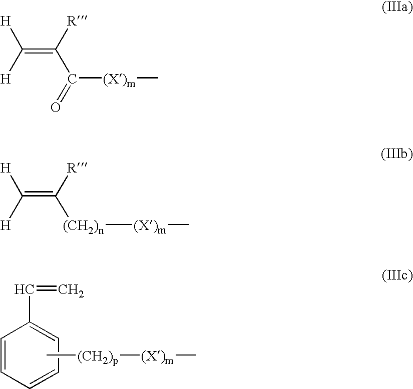
wherein:
R′″ is chosen from hydrogen and linear and branched, saturated C₁-C₆ hydrocarbon-based radicals,
X′ is chosen from O, NH, and NR″″, wherein R″″ is chosen from C₁-C₆ alkyl, C₆-C₁₀ aryl, (C₆-C₁₀)aryl(C₁-C₆)alkyl, and (C₁-C₆)alkyl(C₆-C₁₀)aryl radicals, wherein the alkyl and aryl groups may be substituted with at least one substituent chosen from OH, halogen atoms, and C₁-C₆ alkoxy and C₆-C₁₀ aryloxy radicals;
m is equal to 0 or 1;
n is equal to 0 or 1; and
p is equal to 0, 1 or 2.
